(19) United States
(12) Patent Application Publication
(10) Pub. No.: US 2026/0137829 A1
(43) Pub. Date: May 21, 2026

(54) METHOD FOR DYEING SUTURE MATERIAL

(71) Applicant: Corza Medical, Inc., Boulder, CO (US)

(72) Inventors: Ali Berberogullari, Taunton (GB); Aidan William Nicholson, Taunton (GB)

(21) Appl. No.: 19/395,569

(22) Filed: Nov. 20, 2025

Related U.S. Application Data
(60) Provisional application No. 63/723,492, filed on Nov. 21, 2024.

(51) Int. Cl. A61L 17/04; A61L 17/14; D02G 3/44; D06M 13/152; D06M 15/27; D06M 101/34; D06P 1/36; D06P 3/24

(52) U.S. Cl. CPC A61L 17/04; A61L 17/14; D02G 3/448; D06M 13/152; D06M 15/27; D06P 1/36; D06P 3/245; D06M 2101/34; D10B 2331/02; D10B 2509/04

(57) ABSTRACT

Methods for dyeing suture material that include incubating the suture material in a high temperature staining bath comprising hematein are disclosed. The staining bath may include a nonionic surfactant. The method is carried out in the absence of chromium salts. Also disclosed are (i) a dyed suture material produced by the methods and (ii) a surgical suturing kit that includes the dyed suture material.

### RELATED APPLICATIONS

This application claims priority to U.S. Provisional Patent Application No. 63/723,492 having a filing date of Nov. 21, 2024. The entire disclosure of that application including appendix materials is incorporated herein by reference in its entirety.

### FEDERALLY SPONSORED RESEARCH OR DEVELOPMENT

Not applicable.

### BACKGROUND OF THE INVENTION

The confines created by the US 21 Code of Federal Regulations provide a short list of recognized and approved colorants for surgical sutures in the US market. For nylon and silk sutures, only one dye is approved, i.e., logwood black (“hematein”). This is a historic mordant type of dye. Mordant dyes require a binding agent or mechanism to ensure wash fastness and to determine the hue or final colour.

The principle mordant used for achieving a dark black colour using hematein has been a chrome salt mordant, specifically sodium dichromate. Using this mordant achieves exceptional wash fastness and strong blacks for silk and nylon sutures. Chromates have been phased out of use due to toxicity and environmental concerns.

Alternatives to chromate salts, for example, alum, ferrous ion, and tannins, have been proposed as alternatives for achieving acceptable black shades. However, none of these options are able to achieve the desired black or dark colour using hematein with nylon.

Trials using hematein with a permanganate mordant as an alternative achieved a good colour. However, black particles were released from the dyed material with mechanical agitation.

There is a need to develop methods for coloring suture material using natural dyes in combination with environmentally friendly mordants that yield sufficient dark shades and colour fastness.

### SUMMARY OF THE INVENTION

To meet the need set forth above, methods for dyeing suture material are provided that include incubating the suture material in a heated staining bath comprising hematein. In some embodiments, the methods include dyeing suture material with a pressure dyer, the methods comprising incubating the suture material in a dye solution comprising hematein in a vessel at a high temperature suitable for the suture material used (e.g., at least 110° C., and preferably between 110 and 135° C. or 140° C.) for at least 45 minutes. In some embodiments, the dye solution further comprises a nonionic surfactant and may include a step of mixing hematein and a nonionic surfactant to obtain a dye solution. The methods are carried out in the absence of mordants such as chromium salts.

In some embodiments, the method includes the steps of (a) mixing hematein and a nonionic surfactant to obtain a dye solution; (b) placing a suture material in H₂O in a vessel; (c) heating the H₂O in the vessel from 20° C. to 40° C.; (d) adding the dye solution to the heated H₂O in the vessel to form a staining bath; and/or (e) incubating the suture material in the staining bath at 40° C. to 135° C. for 120 min. to 150 min. The method is carried out in the absence of chromium salts.

In some methods, the methods for dyeing suture material comprise loading the suture material in a vessel in a pressure dyer; adding a staining bath comprising hematein and a nonionic surfactant; increasing the temperature within the vessel to a high temperature; incubating the suture material within the staining bath for at least 45 minutes; and draining the staining bath. The methods are carried out in the absence of mordants such as chromium salts.

In some embodiments, the suture material is silk, nylon, polyester, in construction as a monofilament, e.g., monofilament nylon, multifilament, e.g., multifilament nylon, or braid and/or is in the form of a skein or a roll. In some embodiments, 0.1 kg to 4 kg of the suture material is placed in the vessel. In some embodiments, the dyed suture is nylon having a size of USP 6/0 to USP 2.

In some embodiments, the nonionic surfactant is an ethoxylated castor oil, a polysorbate, a sorbitan ester, a polyoxyethylene alkyl ether, an octylphenol ethoxylate, a block copolymer of ethylene oxide and propylene oxide, an ethoxylated fatty alcohol, a nonylphenol ethoxylate, and/or PEG-36 castor oil.

In some embodiments, a weight to volume ratio between the hematein and the nonionic surfactant is 2.5 kg/L to 12.5 kg/L. In some embodiments, a weight ratio between the hematein and the suture material is 1:1 to 1:6.

In some embodiments, the mixing step further comprises adding at least 4 to 15 volumes of H₂O to dilute the dye solution (staining bath) including prior to step d).

In some embodiments, the incubating is carried out by heating the staining bath from 40° C. to 135° C. over a 30 min period and holding the temperature at 135° C. 5 for 60 min to 120 min.

In some embodiments, the method further comprises the steps of removing the staining bath from the vessel, filling the vessel with H₂O at a temperature of 20° C. to 60° C., adding NaOH to the H₂O in the vessel, maintaining the temperature in the vessel at 40° C. to 60° C. for a period of 30 min to 45 min, draining the vessel, washing the dyed suture material with H₂O, and/or drying the dyed suture material.

In some embodiments, the method further comprises washing the suture material in water at a first temperature less than the incubation temperature and further subjecting the suture material to at least one of (i) a solution comprising sodium hydroxide and/or (ii) a first water rinse at a second temperature greater than the first temperature and a second water rinse at a temperature less than the second temperature. In some embodiments, the method further comprises washing the suture material in water at a temperature that is lower than the incubation temperature or incubating the suture material in a scouring solution.

Also provided are (i) a dyed suture material produced by the disclosed methods and (ii) a surgical suturing kit that includes the dyed suture material and one or more suture needles or other medical devices in a sterilizable package having a plurality of discrete compartments.

As described herein, the novel methods produce suture materials with appropriate diameter, strength, hematein content, and colour characteristics including as described herein without any mordants such as chromium salts. For example, the novel dyed suture materials contain less than 1% hematein and no mordants such as chromium salts (or others discussed herein), achieve sufficient suture strength (e.g., as evaluated using knot pull testing methods), and/or achieve sufficient colour fastness (e.g., as evaluated using colour extractable testing methods).

The details of one or more embodiments of the invention are set forth in the drawings and description below. Other features, objects, and advantages of the invention will be apparent from the description, from the drawings, from the appendices provided with U.S. Provisional Patent Application No. 63/723,492 and hereby incorporated by reference, and from the appended claims.

### DESCRIPTION OF THE DRAWINGS

The patent or application file contains at least one drawing executed in color. It is understood that copies of this patent or patent application publication with color drawing(s) will be provided by the Office upon request and payment of the necessary fee.

FIG. 1 shows photographs of 3.3 kg EP3 (USP 2-0) suture dyed with 0.4 kg hematein for 120 minutes.

FIG. 2 shows photographs of 5.34 kg EP3 (USP 2-0) suture dyed with 2 kg hematein for 120 minutes.

FIG. 3 shows photographs of 3.77 kg EP3 (USP 2-0) suture dyed with 1 kg hematein for 120 minutes.

FIG. 4 shows photographs of 6.72 kg EP3 (USP 2-0) suture dyed with 0.5 kg hematein for 120 minutes.

FIG. 5 shows photographs of 3.88 kg EP3 (USP 2-0) suture dyed with 0.75 kg hematein for 60 minutes.

FIG. 6 shows photographs of 16 packages of EP0.7 to EP1.5 (USP 6-0 to 4-0) suture dyed with 1.25 kg hematein for 45 minutes.

FIG. 7 shows photographs of 3.41 kg EP3 (USP 2-0) suture dyed with 1.25 kg hematein for 90 minutes.

FIG. 8 is a photograph of the tensile strength knot pull Instron testing machine.

FIG. 9A is a box plot of knot pull (KP) strength for EP 3 nylon suture before and after dyeing.

FIG. 9B is a box plot of diameter (DIA) of EP 3 nylon suture before and after dyeing.

FIG. 10A is a box plot of knot pull strength for EP 3.5 (USP 0) nylon suture before and after dyeing.

FIG. 10B is a box plot of diameter of EP 3.5 nylon suture before and after dyeing.

FIG. 11A is a box plot of knot pull strength for EP 4 (USP 1) nylon suture before and after dyeing.

FIG. 11B is a box plot of diameter of EP 4 nylon suture before and after dyeing.

FIG. 12A is a box plot of knot pull strength (MAX.LOAD kgf) for EP 0.7 nylon suture after dyeing shown with historic knot pull strength.

FIG. 12B is a box plot of diameter in mm of EP 0.7 nylon suture after dyeing shown with historic diameter.

FIG. 13A is a box plot of knot pull strength (in kgf) for EP 1 nylon suture after dyeing shown with historic knot pull strength.

FIG. 13B is a box plot of diameter in mm of EP 1 nylon suture after dyeing shown with historic diameter.

FIG. 14A is a box plot of knot pull strength (in kgf) for EP 2 nylon suture after dyeing shown with historic knot pull strength.

FIG. 14B is a box plot of diameter in mm of EP 2 nylon suture after dyeing shown with historic diameter.

FIG. 15A is a box plot of knot pull strength (in kgf) for EP 3 nylon suture after dyeing shown with historic knot pull strength.

FIG. 15B is a box plot of diameter in mm of EP 3 nylon suture after dyeing shown with historic diameter.

FIG. 16A is a box plot of knot pull strength (in kgf) for EP 3.5 nylon suture after dyeing shown with historic knot pull strength.

FIG. 16B is a box plot of diameter in mm of EP 3.5 nylon suture after dyeing shown with historic diameter.

FIG. 17A is a box plot of knot pull strength (in kgf) for EP 5 nylon suture after dyeing shown with historic knot pull strength.

FIG. 17B is a box plot of diameter in mm of EP 5 nylon suture after dyeing shown with historic diameter.

FIG. 18A is a Process Capability Report of knot pull strength for EP 3 nylon suture after dyeing.

FIG. 18B is a Process Capability Report for EP 3 nylon suture after dyeing.

FIG. 19A is a Process Capability Report of knot pull strength for EP 3.5 nylon suture after dyeing.

FIG. 19B is a Process Capability Report for EP 3.5 nylon suture after dyeing.

FIG. 20A is a Process Capability Report of knot pull strength for EP 4 nylon suture after dyeing.

FIG. 20B is a Process Capability Report for EP 4 nylon suture after dyeing.

FIG. 21A is a Process Capability Report of knot pull strength for EP 0.7 nylon suture after dyeing.

FIG. 21B is a Process Capability Report for EP 0.7 nylon suture after dyeing.

FIG. 22A is a Process Capability Report of knot pull strength for EP 1 nylon suture after dyeing.

FIG. 22B is a Process Capability Report for EP 1 nylon suture after dyeing.

FIG. 23A is a Process Capability Report of knot pull strength for EP 2 nylon suture after dyeing.

FIG. 23B is a Process Capability Report for EP 2 nylon suture after dyeing.

FIG. 24A is a Process Capability Report of knot pull strength for EP 3 nylon suture after dyeing.

FIG. 24B is a Process Capability Report for EP 3 nylon suture after dyeing.

FIG. 25A is a Process Capability Report of knot pull strength for EP 3.5 nylon suture after dyeing.

FIG. 25B is a Process Capability Report for EP 3.5 nylon suture after dyeing.

FIG. 26A is a Process Capability Report of knot pull strength for EP 5 nylon suture after dyeing.

FIG. 26B is a Process Capability Report for EP 5 nylon suture after dyeing.

FIG. 27 is a photograph of a roll of nylon suture dyed with the prior art dichromate process.

FIG. 28 is a photograph of 2 reels taken from a dyeing run carried out with 2 kg of hematein on the left and a dyeing run carried out with 0.5 kg of hematein on the right.

### DETAILED DESCRIPTION OF THE INVENTION

Described herein are methods for dyeing suture material. The methods include incubating the suture material in a staining bath comprising hematein heated to a high temperature for at least 45 minutes in the absence of mordants such as chromate salts. In some embodiments, the staining bath further comprises a nonionic surfactant. In some embodiments, the methods can include the steps of (a) mixing hematein and a nonionic surfactant to obtain a dye solution; (b) placing a suture material in H₂O in a vessel; (c) heating the H₂O in the vessel from 20° C. to 40° C.; (d) adding the dye solution to the heated H₂O in the vessel to form a staining bath; and/or (e) incubating the suture material in the staining bath at 40° C. to 135° C. for 120 min. to 150 min. The methods are carried out in the absence of chromium salts. The methods can be carried out in a pressure dyer vessel. Also provided are (i) a dyed suture material produced by the disclosed methods and (ii) a surgical suturing kit that includes the dyed suture material and other materials. As described herein, the methods produce dyed suture materials with appropriate diameter, strength, hematein content, and colour characteristics.

In some aspects, the suture material is preferably non-resorbable and can be, but is not limited to, silk, nylon, or polyester, e.g., polyethylene terephthalate (“PET”). In a particular method, the suture material is nylon, for example, monofilament nylon and multifilament nylon. The suture material can be a suture of any size known in the art, e.g., EP 0.01, 0.1, 0.2, 0.3, 0.4, 0.5, 0.7, 1.0. 1.5. 2, 3, 3.5, 4, 5, 6, 7, 8, 9, 10, 11, and 12, corresponding to USP 12-0, 11-0, 10-0, 9-0, 8-0, 7-0, 6-0, 5-0, 4-0, 3-0, 2-0, 1-0, 0, 1, 2, 3 and 4, 5, 6, 7, 8, 9, and 10, respectively. The suture material can be in the form of a skein, i.e., loosely coiled. Alternatively, the suture material can be in the form of a roll, e.g., wound around a dye package tube. The dye package tube can be a perforated plastic tube that can withstand elevated temperatures of at least 135° C. during the dyeing process. Exemplary dye package tubes are formed of polypropylene and can be rigid or flexible. The dye package tube can have a length of 150 mm to 300 mm and an inside diameter of 50 mm to 70 mm. Each roll can include 0.1 to 1 kg of suture material.

In some embodiments, the method for dyeing suture material requires mixing hematein with a nonionic surfactant. The nonionic surfactant can be, but is not limited to, an ethoxylated castor oil, a polysorbate, a sorbitan ester, a polyoxyethylene alkyl ether, an octylphenol ethoxylate, a block copolymer of ethylene oxide and propylene oxide, an ethoxylated fatty alcohol, and a nonylphenol ethoxylate.

Exemplary nonionic surfactants include Tween Series (Polysorbates), e.g., Tween 20-Tween 40, Tween 60 Tween 80, (Polyoxyethylene (20) sorbitan monolaurate); Span Series (Sorbitan Esters), e.g., Span 20, Span 40, Span 60, Span 80 (Sorbitan monolaurate); Brij Series (Polyoxyethylene Alkyl Ethers), e.g., Brij 30, Brij 35, Brij 58, Brij 72,76 (Polyoxyethylene); Triton Series (Octylphenol Ethoxylates), e.g., Triton X-100, Triton X-114 (Octylphenol ethoxylate); Pluronic Series (Block Copolymers of Ethylene Oxide and Propylene Oxide), e.g., Pluronic L61, Pluronic F68, Pluronic P123, Pluronic F127; Ethoxylated Fatty Alcohols C12-C15, e.g., Pareth-7 (Ethoxylated C12-C15 alcohol with 7 EO), Ceteareth-20 (Ethoxylated cetyl/stearyl alcohol with 20 EO); and Nonylphenol Ethoxylates NP-9 (Nonylphenol ethoxylate, 9 moles EO) & NP-10 (Nonylphenol ethoxylate, 10 moles EO).

In certain methods, the nonionic surfactant is an ethoxylated castor oil that can be, but is not limited to, PEG-5 Castor Oil: Castor oil ethoxylated with 5 moles of ethylene oxide; PEG-10 Castor Oil: Castor oil ethoxylated with 10 moles of ethylene oxide; PEG-20 Castor Oil: Castor oil ethoxylated with 20 moles of ethylene oxide; PEG-30 Castor Oil: Castor oil ethoxylated with 30 moles of ethylene oxide; PEG-35 Castor Oil: Castor oil ethoxylated with 35 moles of ethylene oxide; PEG-36 Castor Oil: Castor oil ethoxylated with 36 moles of ethylene oxide; PEG-40 Castor Oil: Castor oil ethoxylated with 40 moles of ethylene oxide; PEG-50 Castor Oil: Castor oil ethoxylated with 50 moles of ethylene oxide; and PEG-60 Castor Oil: Castor oil ethoxylated with 60 moles of ethylene oxide.

In a particular method, the nonionic surfactant is BEROL® 904 (PEG-36 Castor Oil).

The method may be carried out within a certain weight to volume ratio between hematein and the nonionic surfactant, e.g., BEROL® 904. The weight to volume ratio between the hematein and the nonionic surfactant can be from 2.5 kg hematein/liter of nonionic surfactant up to 12.5 kg/L (e.g., 2.5, 3, 3.5, 4, 4.5, 5, 5.5, 6, 6.5, 7, 7.5, 8, 8.5, 9, 9.5, 10, 10.5, 11, 11.5, 12, and 12.5 kg/L).

In a particular method, a mixture of hematein and nonionic surfactant, i.e., dye solution, is diluted by adding at least 4 to 15 volumes of H₂O (e.g., 4, 5, 6, 7, 8, 9, 10, 11, 12, 13, 14, or 15) to form the staining bath prior to incubation with the suture material.

The amount of hematein used in the method can be adjusted depending upon the amount of suture material to be dyed. For example, the weight ratio of hematein to suture material (e.g., nylon) can range from 1:1 to 1:6 (e.g., 1:1, 1:1.5, 1:2, 1:2.5, 1:3, 1:3.5, 1:4, 1:4.5, 1:5, 1:5.5, and 1:6).

In some aspects, the amount of suture material that can be dyed in a single batch by the method can vary over a large range. Exemplary methods can effectively dye from 0.1 kg to 4 kg (e.g., 0.1 kg, 0.25 kg, 0.5 kg, 0.75 kg, 1 kg, 1.25 kg, 1.5 kg, 1.75 kg, 2 kg, 2.25 kg, 2.5 kg, 2.75 kg 3 kg, 3.25 kg, 3.5 kg, 3.75 kg, and 4 kg) of suture material in a single batch.

To achieve acceptable staining of the suture material, it is incubated in a vessel (e.g., vat or container) together with the staining bath (dye solution) at a high temperature suitable for the suture material being used. In some methods, incubation occurs at 40° C. to 135° C. for 120 min. to 150 min. In a particular method, during the incubation period, the staining bath is heated from 40° C. to 135° C. over a 30 min. period and the temperature held at 135° C. for 60 min to 120 min (e.g., 60 min., 75 min., 90 min., 105 min., or 120 min.). Alternatively, the staining bath can be heated from 40° C. to 135° C. at a rate of 1° C./min. to 3.5° C./min. (1, 1.5, 2, 2.5, 3, and 3.5° C./min).

The methods for dyeing suture material described above can include the following additional steps: (f) removing the staining bath from the vessel; (g) filling the vessel with H₂O at a temperature of 20° C. to 60° C. (e.g., 20° C., 25° C., 30° C., 35° C., 40° C., 45° C., 50° C., 55° C.); (h) adding NaOH to the H₂O in the vessel; (i) maintaining the temperature in the vessel at 40° C. to 60° C. for a period of 30 min. to 45 min.; (j) draining the vessel; (k) washing the dyed suture material with H₂O; and/or (l) drying the dyed suture material.

In a particular method, the amount of H₂O added in step (g) is the maximum amount of volume that can be added to the vessel. In step (h), NaOH, i.e., caustic soda, is added at a ratio of 1 part by volume NaOH to 25-75 parts by volume H₂O (e.g., 1:25, 1:30, 1:35, 1:40, 1:45, 1:50, 1:55, 1:60, 1:65, 1:70, and 1:75).

A particular method includes drying step (I) that is accomplished by incubating the dyed, washed suture material in a warm, humidity-controlled environment. For example, the suture material can be kept at 30° C. to 37° C. in 50% humidity for a period of 12-36 h. In an exemplary, method, the suture material is dried at 34° C. at 50% humidity for 24 h.

In one aspect, the invention includes methods for dyeing suture material with a pressure dyer, the method comprising incubating the suture material in dye solution in a vessel at a high temperature for at least 45 minutes wherein the method is carried out in the absence of mordants such as chromium salts. In some methods, the dye solution or staining bath further comprises a nonionic surfactant and may include a step of mixing hematein and a nonionic surfactant to obtain a dye solution or staining bath prior to incubation. In some methods, the methods for dyeing suture material include loading the suture material in a vessel in a pressure dyer, adding a staining bath comprising hematein and a nonionic surfactant (without the addition of mordants such as chromate salts) to the vessel, increasing the temperature within the vessel comprising the suture material and staining bath to a high temperature (preferably to between about 110 and 135° C. or 140° C.), incubating the suture material within the staining bath at the high temperature for at least 45 minutes (e.g., 45 to 150 minutes), and/or draining the staining bath. In some embodiments, the suture material is further subjected to: a water rinse at a cooler temperature, e.g., 40° C.; a solution comprising sodium hydroxide (which may be heated); a water rinse at a warmer temperature, e.g., 60° C.; and a water rinse at a cooler temperature, e.g., 40° C.

In some methods, a pressure dyer is loaded with nylon skeined or packaged suture material and is subjected to a programmed process to achieve the dyed suture material. The vessel is filled with water and brought to a standardized temperature, e.g., 20° C. to 40° C. (including individual temperatures within that range, e.g., 25° C., 30° C., 35° C., etc.). A premixed solution comprising hematein and a nonionic surfactant are added. The dye solution does not include typical mordants, e.g., chromate salts. The temperature of the vessel is increased to a predetermined temperature between 40° C. to 135° C. or 140° C. (preferably to a suitable high temperature above about 110° C., 115° C., 120° C., 125° C., 130° C., 135° C., etc.). The suture material is incubated in the high temperature staining bath for at least 45 minutes, e.g., between 45 to 120 minutes or 120 minutes to 150 minutes (including individuals times within that range, e.g., 50, 55, 60, 65, 70, 75, 80, 85, 90, 95, 100, 105, 110, 115, 120, 125, 130, 135, 140, 145, 150, 155, etc.). The vessel is drained.

In some embodiments, the suture material within the pressure dyer can be subjected to a “cold” rinse at a temperature below the predetermined temperature. Water is added to the vessel and the vessel is cooled/heated to the selected temperature (e.g., 40° C.). The water is drained after a period (e.g., 10-15 minutes). In some embodiments, the suture material can be subjected to a solution comprising a scouring material, e.g., a sodium hydroxide solution is added to the vessel. The suture material is incubated in the scouring solution for a period (e.g., about 30 minutes). During this time, the vessel may be heated (e.g., to a temperature of 50° C. or 60° C.). The scouring solution is then drained. In some embodiments, the suture material can be subjected to a “hot” rinse at a temperature greater than the temperature of a “cold” rinse. Water is added to the vessel and heated to the selected temperature (e.g., 60° C.). In some embodiments, the suture material can then be subjected to a “cold” rinse. After completion of the final step utilizing the pressure dyer, all solutions and baths are drained from the vessel and the dyed suture material is removed from the vessel.

Dyed suture material can be sterilized and packaged by any method known in the art.

The dyed suture material can be used advantageously in many types of surgery, including, but not limited to, plastic and reconstructive, cardiovascular, orthopedic, neuro-, ophthalmic, gastrointestinal, pediatric, gynecological, oral, and maxillofacial.

Dyed sutures allow the surgeon to create small incisions and to delicately manipulate the suture, particularly in cases where placement of sutures can be critical to minimize scarring. The high visibility of the dyed suture ensures precise placement to reduce chances of inadvertently pulling the suture through soft tissue or bone. The dyed suture is also beneficial during closure of deep tissue layers or when using a subcuticular technique. Dyed sutures have enhanced visibility that facilitates knot tying by the surgeon. Dark colored sutures permit easier detection of tissue reactions such as inflammation or infection. Finally, dyed sutures are also easier to spot and remove, if needed, with minimal handling.

The dyed suture material can be packaged as is or packaged and used within surgery suture kits with needle attachments. For example, the sizes of dyed nylon sutures with needle attachment can be between USP 6/0 to USP 2.

All of the methods and embodiments thereof described above are carried out in the absence of typical mordants, including but not limited to chromium salts, tannic acid, oxalic acid, alum, chrome alum, sodium chloride, and salts of aluminum, copper, iron (ferrous sulfate), iodine, potassium, sodium, tungsten, and tin. The use of a nonionic surfactant, e.g., BEROL® 904, allows the hematein to induce a black colour within the nylon without the use of chromate salts as a mordant. Not to be bound by theory, it is believed that the mechanism by which this takes place is due to the reduction in surface tension of water. Water has a high surface tension due to strong hydrogen bonding between the molecules. For example, when surfactants are added to water, they provide a lower intermolecular force than that between water molecules due to their amphipathic properties (having both hydrophilic and hydrophobic parts). This lowers the surface and interfacial tension between the molecules and permits the hematein to penetrate the nylon.

Without further elaboration, it is believed that one skilled in the art can, based on the disclosure herein, utilize the present disclosure to its fullest extent. The specific examples discussed herein are, therefore, to be construed as merely descriptive, and not limitative of the remainder of the disclosure in any way whatsoever. All publications and patent documents cited herein are incorporated by reference in their entirety.

### Examples

In one engineering study documented in U.S. Provisional Patent Application No. 63/723,492 Appendix A, the process of dyeing monofilament black nylon using an alternative process to the historical method of sodium dichromate mordant is documented prior to an operational and performance qualification validation process.

As discussed above, historically, the method of Logwood Dye and Sodium Dichromate as a mordant has been utilized in the production of dyed monofilament nylon black. Due to concerns regarding the potential carcinogenic properties of chromates, a new process is investigated to replace this historic method. Using permanganate mordant as an alternative provided initial positive potential. It achieves a good colour, passing both the hematein and extractable colour testing. However, was unsuccessful and not taken further due to findings with the “rub test” whereby black particles are mobile under agitation, and efforts to resolve this issue were unsuccessful.

This study documents new alternative methods to produce dyed black nylon that provides sufficient diameter, strength, extractable colour, and hematein content. The process in this study uses Thies 500-01 pressure dyeing machine with hank and package frames, packaged nylon sizes EP 0.7 to EP 2, and/or skeined Nylon sizes EP 3 to EP 5. Skeins of monofilament nylon 6.6 of size EP3 and packages of size EP 0.7 to 1.5 are selected as representatives for the purposes of the study. It is noted that The Thies 500-01 ECO BLOCK consists of the machine component (Theis 500-01, pressure dyeing vessel), software component (Sedomat 2500, touch screen controller, displays process steps, and machine status), and hank frame (frame for dyeing 2-4 kg of hank (skein) material).

The process conducted for this study is shown in U.S. Provisional Patent Application No. 63/723,492 Appendix C. An overview of the process includes the following steps: (1) Load the Thies 500 pressure dyer with Nylon skeined or packaged material; (2) Fill vessel with water and insert a premixed quantity of Hematein HCK S21 and Berol 904 at 1352C; (3) Drain vessel; (4) Fill vessel with water and insert Caustic Soda (Sodium Hydroxide) at 60° C.; (5) Water rinses at hot and cold temperatures; (6) Process completes.

Further details regarding the process (subject to other process conditions discussed herein, e.g., temperature and time) are shown in Table 1 below.

TABLE 1

| | Step | | |
|---|---|---|---|
| Bath No. | No. | Process | Time |
| 1 | 01 | Start | |
| Dye Run | 02 | Pump set | 5 |
| | 03 | Load machine | 5 |
| | 04 | Fill level 95% | 5 |
| | 05 | Heat/cool machine (m/c) 40° C., Fill | 5 |
| | | addition tank (AT) 20% | |
| | 06 | Mix chemicals within addition tank (AT) | 10 |
| | | Hematine 1000 gr. | |
| | | Berol 904 280 ml | |
| | 07 | Dosing AT, Fill stock tank (ST) | 5 |
| | 08 | Heat/cool m/c at 135° C. | 120 |
| | 09 | Drain m/c | 5 |
| 2 | 10 | Fill m/c, ST to m/c 95% | 5 |
| Cold Rinse | 11 | Heat/cool m/c, Fill ST 72% 40° C. | 10 |
| | 12 | Drain m/c | 5 |
| 3 | 13 | ST to m/c 95% | 5 |
| Scour | 14 | Heat/cool m/c at 50° C., Fill AT 25% | 1 |
| | 15 | Add caustic soda 500 ml | 5 |
| | 16 | Heat/cool m/c at 60° C., Fill ST 72% | 30 |
| | | 40° C., Dosing AT | |
| | 17 | Drain m/c | 5 |
| 4 | 18 | ST to m/c 100% | 5 |
| Hot Rinse | 19 | Heat/cool m/c at 60° C., Fill ST | 10 |
| | 20 | Drain m/c | 5 |
| 5 | 21 | ST to m/c | 5 |
| Cold Rinse | 22 | Heat/Cool - Cold Rinse | 5 |
| | 23 | Drain m/c, Drain ST | 5 |
| | 24 | Program end | |

Validation in the form of an operational qualification followed by a performance qualification is used to fully validate the process. This can be done on the Thies 500-01 pressure dyer and using packages for sizes EP 0.7 to EP 2, as well as skeins of sizes EP 3 to EP 5.

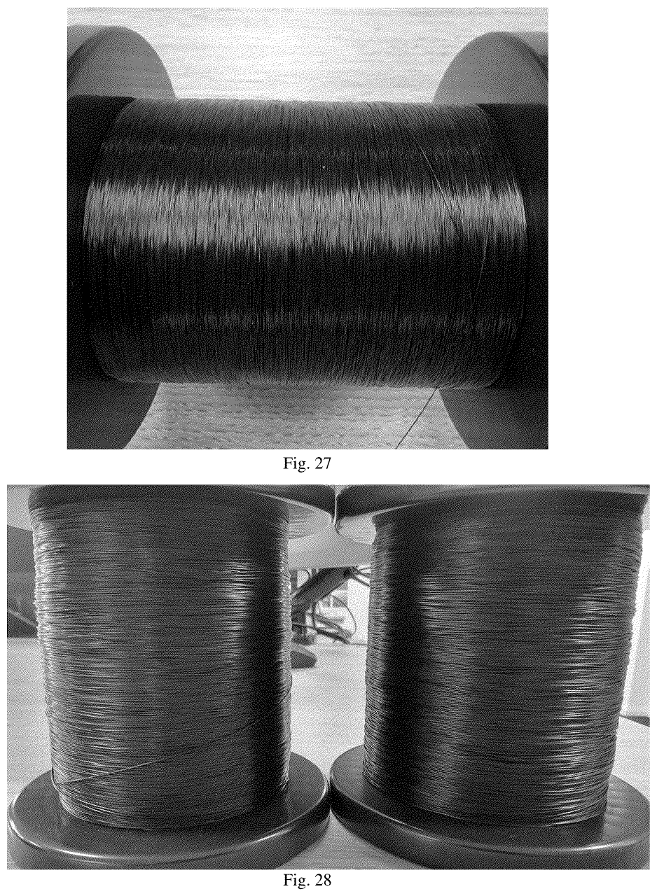

Seven separate trial runs are completed for this study. The trials utilize the following variables: dye concentrations between 0.5 kg and 2 kg of Hematein, duration of dye process between 45 minutes and 120 minutes, and quantity of nylon used. The process involves the use of the following fixed quantities: 200 ml of Berol and 500 ml of Caustic Soda. The control for which to compare this new process to is the historic sodium dichromate process. FIG. 27 is a photograph of a roll of nylon suture dyed with the prior art dichromatic process.

As shown in Table 2, the following seven trials are completed using the process with changed variables.

TABLE 2

| Trial | | Hematein | Duration of | Quantity of |
|---|---|---|---|---|
| Number | Size | Quantity | Dye run | Nylon |
| 1 | Skein (EP3) | 0.4 kg | 120 minutes | 3.3 kg |
| 2 | Skein (EP3) | 2 kg | 120 minutes | 5.34 kg |
| 3 | Skein (EP3) | 1 kg | 120 minutes | 3.77 kg |
| 4 | Skein (EP3) | 0.5 kg | 120 minutes | 6.72 kg |
| 5 | Skein (EP3) | 0.75 kg | 60 minutes | 3.88 kg |
| 6 | Skein (EP3) | 1.25 kg | 90 minutes | 3.41 kg |
| 7 | Packages (EP 0.7 | 0.5 kg | 45 minutes | 16x Packages |
| | to EP1.5) | | | |

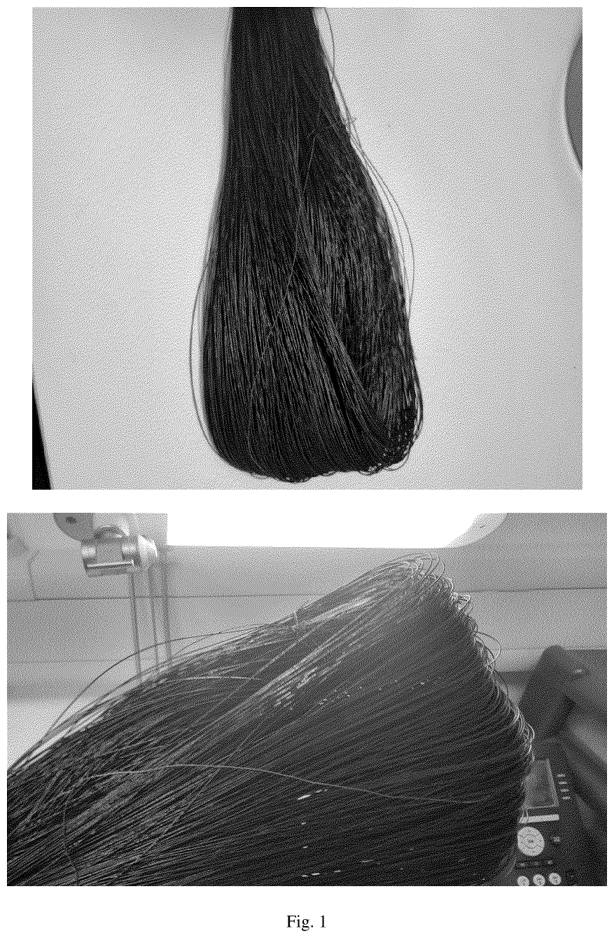

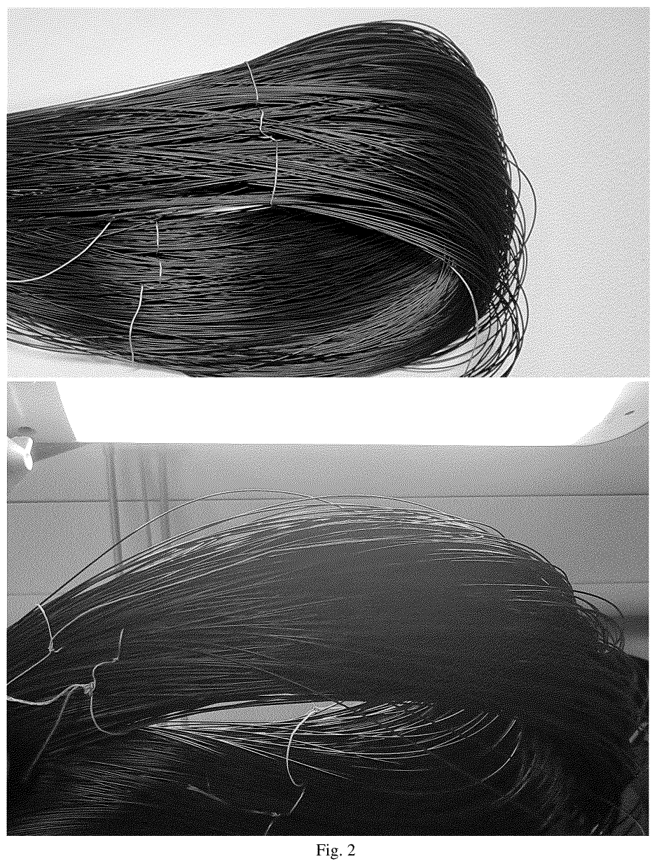

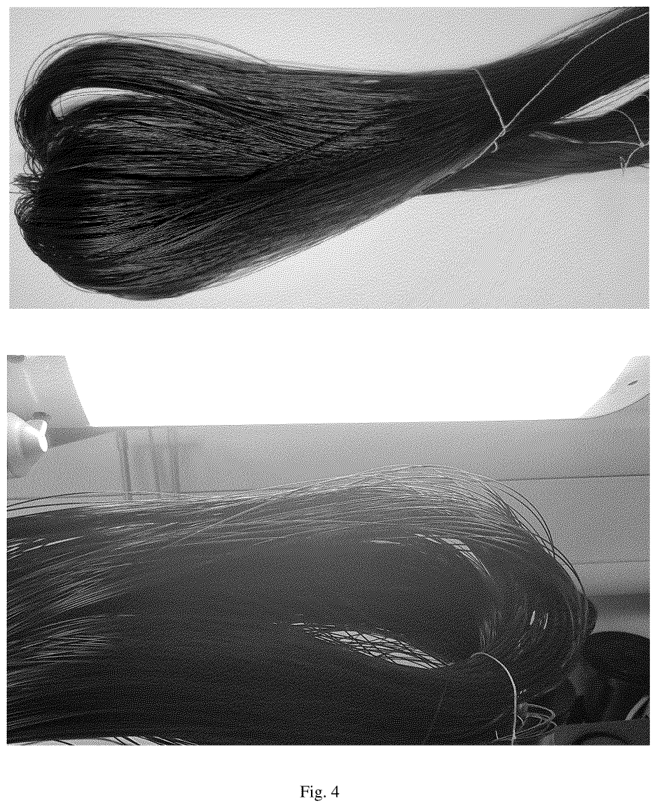

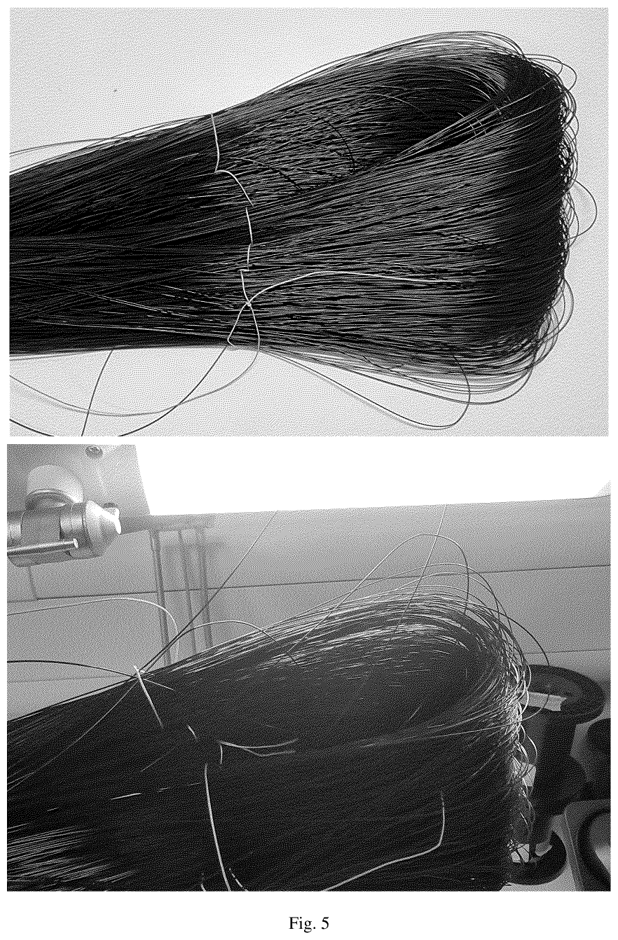

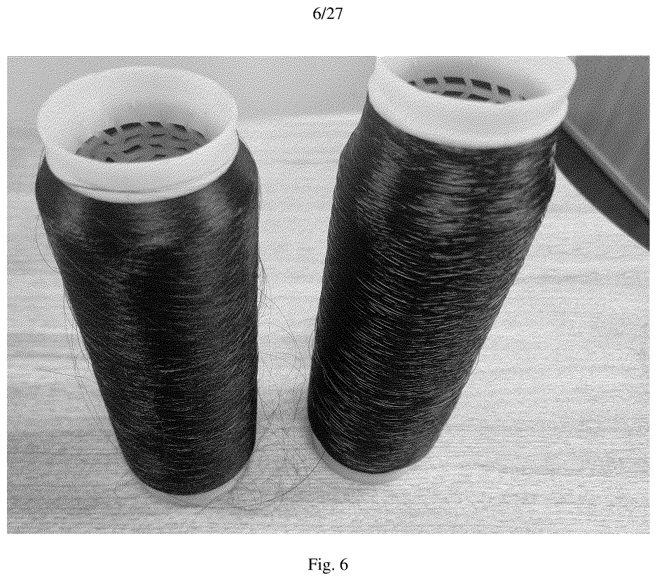

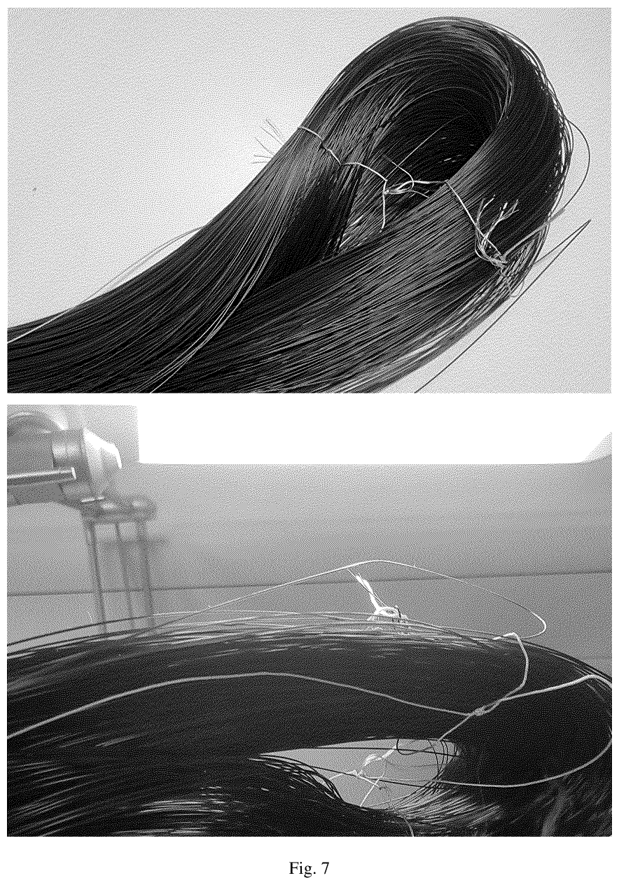

FIGS. 1, 2, 3, 4, 5, 6, and 7 depict photographs of dyed sutures from Trials 1-7. FIG. 28 shows an example of 2 reels taken from the second run with 2 kg of hematein on the left and from the fourth run with 0.5 kg of hematein on the right.

Testing of hematein and extractable colour testing is conducted for the second run using 2 Kg of hematein, passing with an average % hematein of 0.23%, below the tolerance of 1%. And, as discussed below, for example, in an extractable colour testing method using a spectrophotometer, passing with <1.00 (370 nm) and <0.1 (500 nm). As this is the largest dye quantity used, it is approximated that lower concentrations of dye will also pass this testing. This data is provided in U.S. Provisional Patent Application No. 63/723,492 Appendix B, e.g., showing 370 nm (0.059) and 500 nm (0.043).

Diameter testing of US EP3 skeins before and after concludes that the mean diameter slightly increased from 0.33 to 0.34 mm. All values remained within specification for EP3 (0.30 to 0.349 mm).

Knot pull testing for EP3 skeins post dyeing has a mean of 2.5 KgF, above tolerance specification for EP3 of 1.8 KgF.

As can be seen in FIGS. 1-7, good colour fastness can be observed for higher concentrations of hematein above 1 kg. Observing the nylon with a light source behind the material at lower concentrations of hematein the colour can shift to a red/brown, which is also satisfactory. It can also be seen from the duration of dye run that 120 minutes is not required to achieve an acceptable colour fastness, with material being sufficiently dyed at durations lower than 120 minutes.

Cleaning the material using caustic soda achieves good results. The issue observed during the potassium permanganate trials during the “rub-test” is no longer an issue, and the material has a tactile feel resembling that which is expected for monofilament nylon.

The process in the study provides good evidence that monofilament nylon can be sufficiently dyed without the use of sodium dichromate. The material has a visible likeness to the historic method while passing the hematein and extractable colour testing.

Extractable colour testing methods including Logwood black silk dyed is provided in U.S. Provisional Patent Application No. 63/723,492 Appendix E. The testing ensures that extractable colour does not exceed level determined by depth of colour of extract against USP matching solutions. Inventory includes conical flask, hot plate, measuring glass, de-ionised water, spectrophotometer, and colour samples. The test procedure is as follows. 1. Prepare 250 ml of deionised water in a heat-resistant flask covered with a short stemmed funnel and place on hot plate at 370° and bring water to boil. 2. Weigh 2.5 g of suture and place into the flask once the water has started to gently boil. Turn temperature down to 270° and gently boil the suture and water for 15 minutes. 3. Remove the suture, turn off the hot-plate and restore water level up until each 10 mg of suture that was initially added is diluted by 1 ml of water, place the flask into the fume cupboard for 3 hours to cool until the liquid is at room temperature. 4. Decant deionised water into a 5 ml optical glass cell using a pipette—this will be the control sample. 5. Decant the cooled solution into a different 5 ml optical glass cell using a pipette. 6. Turn on the spectrophotometer and wait until start up sequence is complete. 7. Set the wavelength to 370 nm and place the fresh control sample in the spectrophotometer. Close the spectrophotometer lid and calibrate the spectrophotometer at this wavelength. 8. Remove the control sample and locate the generated sample solution in the spectrophotometer, close the lid and record the observed value. 9. Set the wavelength to 500 nm and place the fresh control sample in the spectrophotometer. Close the spectrophotometer lid and calibrate the spectrometer at this new wavelength. 10. Remove the Control Sample and locate the generated sample solution in the spectrophotometer, close the lid and record the observed value. 11. Refer to Table 3 below for acceptance and referral action (also discussed above). It is noted that Med/PE wax coated material that fails at dye stage 500 nm above 0.14, and untreated and beeswax coated material that fails at dye stage 500 nm above 0.10, should be returned for rinsing. Max number of re-rinses is 3.

TABLE 3

| | | Dye | Refer | Intermediate |
|---|---|---|---|---|
| Suture product | Wavelength | pass | intermediate | if referred |
| untreated | 500 nm | <0.1 | NA | NA |
| wax | 500 nm | <0.1 | NA | <0.1 |
| med and polyethylene | 500 nm | <0.1 | 0.1 to 0.14 | <0.1 |
| (PE) wax | | | | |
| all | 370 nm | <1.00 | NA | NA |

The sample preparation procedure is as follows. Prepare the matching solution that corresponds to the extractable colour by combining the Colorimetric Solutions (CS) in the proportions indicated in Table 4 adding water if necessary to make 10.0 parts. The values in brackets are in ml against each CS for the stated quantity of water. The samples are to be retained out of the light in a suitable storage cupboard and are to be referenced visually as required. The read out from the pink-red solution from the spectrometer should be 0.110 to 0.115 at 500 nm and for yellow-brown 1.8 to 1.9 at 370 nm. Also, the solid colour sample can be used to compare results between spectrometers when set to absorbance and wavelengths as listed on the associated raw data sheet accompanying the gauge.

TABLE 4

| | Parts of each CS per 10 parts total volume | | | |
|---|---|---|---|---|
| Colour of | Red primary | Yellow Primary | Blue Primary | |
| suture | solution | solution | Solution | |
| (extractable | (Cobaltous | (Ferric | (Cupric | Purified |
| colour) | Chloride CS) | Chloride CS) | Sulfate CS) | water |
| Yellow-brown | 0.2 (10) | 1.2 (60) | — | 430 ml |
| Pink-Red | 1.0 (90) | — | — | 90 ml |

A simple knot pull method for testing tensile strength is described in U.S. Provisional Patent Application No. 63/723,492 Appendix D. FIG. 8 is a photograph of a tensile strength knot pull Instron testing machine. The products covered are polyester (standard and SPS, all colours and coatings), silk (all colours and coatings), nylon and polypropylene monofilament (all colours), and/or raw material yarn (all types). Simple knot pull is a main acceptance criteria for the US or European pharmacopoeias apart for small sizes USP9/0 and below. It is performed as part of Pearsalls test plans for standard bulk braid. Elongation (Crosshead) Speed is set at 300 mm/min within the machine software. As shown in FIG. 8, the test is performed at a 5″ Gauge Length (gap between the jaws).

The procedure is as follows. Having taken a length from the bobbin—minimum of 500 mm. Tie simple knot in test length by passing the end held in the right hand over that held in the left hand and drawing the free end through the loop so formed. Fasten test length in the instrument jaws with the knot positioned approximately central between the two jaws. Clamp in place, using the foot plate. Initiate the test through computer software. Allow the test to complete to breaking point. Use the foot plate to release the sample and discard. Repeat for other samples of the same batch. Breaks occurring immediately adjacent to the jaws are to be ignored, and the test to be repeated.

Slippage is when the jaws clamping the material being tested allow the material to travel as the force is being applied during the test. Slippage can cause false results. Therefore, visually monitoring the test is required at all times. Slippage is not dependent upon a particular product, or upon the size being tested, although the frequency of it occurring is higher for sizes of a larger diameter. Also, a particular finish can affect slippage, but even so the variability of slippage occurring cannot be predicted, even if in some instances it is expected. Therefore, all tests must be visually monitored to ensure no false results occur. When slippage does occur, there are several methods that can be used to counterpoise this: wrapping the braid around the jaws of the Instron Machine; using the forceps, twist the braid and hold against the bottom jaw; tie a double loop knot at the base of the top jaw, and also wrap around the jaw to prevent the braid from slipping through the jaws.

Other studies regarding the methods are also undertaken as documented in U.S. Provisional Patent Application No. 63/723,492 Appendices C and F. An operational qualification (OQ) report, documented in U.S. Provisional Patent Application No. 63/723,492 Appendix F, verifies that the production of Black Nylon using the Thies 500 Pressure Dyer (Thies 500-01) operates according to the engineering design specifications and criteria. Specifically, that it can operate effectively across a range of hematein quantities in the production black nylon in skein format to produce acceptably dyed product within specification. The scope of the OQ protocol is limited to Thies 500 Pressure Dyer (Thies 500-01) in the production of Black Nylon USP 2-0 (EP 3), USP 0 (EP 3.5), USP 1 (EP 4)—skein dyeing. The software system is validated in accordance with 21 CFR parts 820.70(i) and ISO13485 Section 4.1.6.

According to the protocol and for validation purposes, the range of nylon that will be dyed in hank format will also include USP 2 (EP 5) and would be from hematein quantities of 1 kg, 1.25 kg and 1.5 kg. USP 2 (EP 5) material is not available during the operational qualification phase and will instead be included within the performance qualification.

The three hematein trials were conducted at 0.75 kg, 1 kg and 1.25 kg due to the good colour uptake at all quantities.

All three dye runs are successful in that a good colour uptake of ‘black’ was achieved. The testing of knot pull remains firmly within specification post dyeing. As shown in Table 5, there is an increase in diameter when compared to the pre-dye tested material of between 4.95 to 7.8% across the sizes tested. This results in the diameter for EP 3 and 3.5 being slightly above average maximum specification as discussed herein

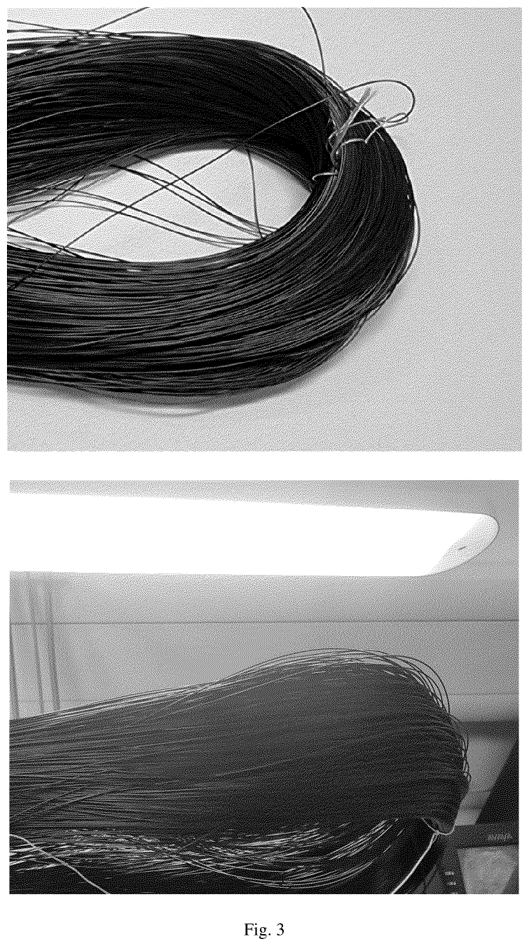

It is noted that, following the three successful dye runs with hematein quantities of 0.75 kg, 1 kg and 1.25 kg in the engineering study (FIGS. 3, 5, and 7), the dye quantity is lowered further to observe a point at which the colour uptake is not adequate. A fourth run is conducted with 0.5 kg of hematein and produces products which mostly have a good colour uptake but do have noticeable areas of brown patches. It is also noticed that on observing with light behind the material that it has a shade of dark brown. See FIG. 4.

Results from the operational qualification are shown in Tables 5, 6, 7, and 8 below. Acceptance criteria for this particular testing is shown in Tables 6, 7, and 8 including product characteristics for diameter (USP) (Acceptance Quality Level (AQL) 0.28%, sample size 40), knot pull (KgF) (AQL 0.31%, sample size 40), extractable colour (sample size 1), and hematein percentage (sample size 1). It is noted that reference measurements are taken for diameter (USP) and knot pull (KgF) (sample size 40). It is further noted that sample sizes reflect minimum samples undertaken for each reference or run.

TABLE 5

| | | | Average | |
|---|---|---|---|---|
| | Pre-Dye | Post-Dye | Maximum | Increase |
| | Mean | Mean | Specification | Pre-Dye to |
| Size | (mm) | (mm) | (mm) | Post Dye % |
| EP3 | 0.3359 | 0.3525 | 0.349 | 4.95% |
| EP3.5 | 0.3783 | 0.4078 | 0.399 | 7.80% |
| EP 4 | 0.4513 | 0.4780 | 0.499 | 5.91% |

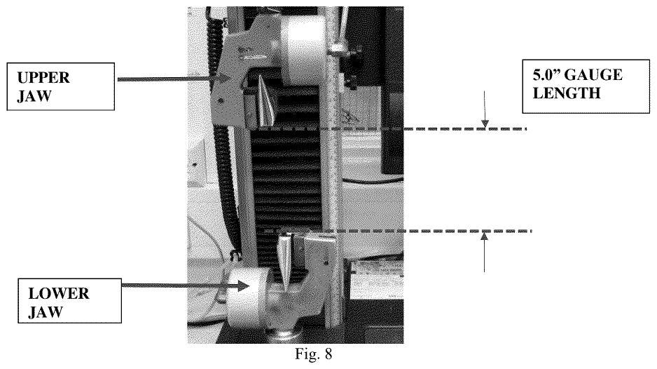

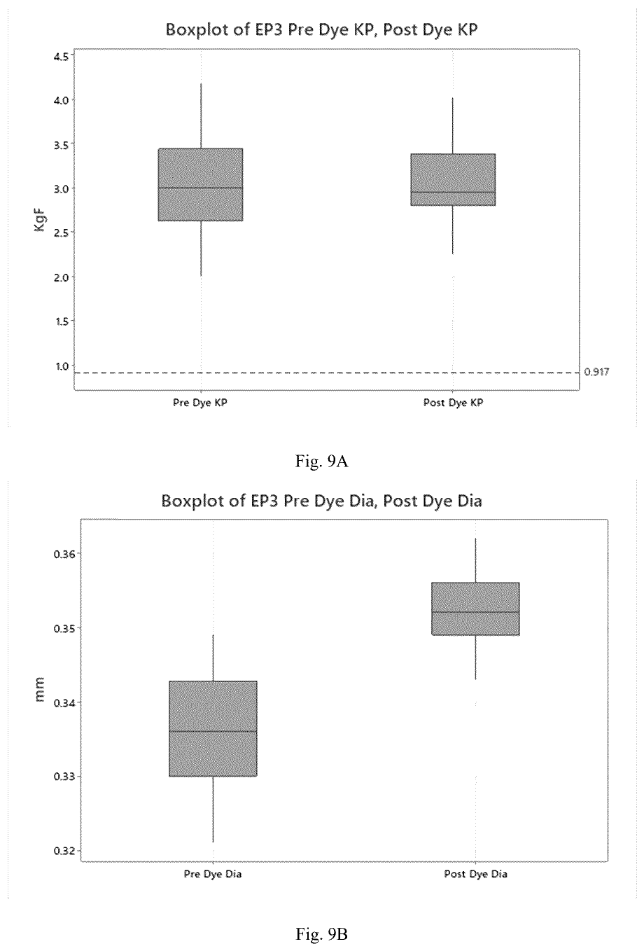

FIGS. 9A and 9B are blox plots of knot pull (KP) strength and diameter (DIA) for EP3 nylon suture before and after dyeing. FIGS. 18A and 18B are process capability reports of knot pull strength and diameter for EP3 nylon suture after dyeing. As is evident from the drawings, other data is reflected in the process capability reports including process data and overall capability including process performance index (PpK).

TABLE 6
Part Number: 70A302000 USP 2-0 (EP 3)

| Product | | | |
|---|---|---|---|
| Characteristic | Acceptance Criteria | Results | Pass/Fail |
| Diameter (USP) | 0.2245 ≤ X ≤ 0.3745 | x̅ = 0.3525 | PASS* |
| | (PpK > 0.92) | Ppk = 1.56 | |
| Knot Pull (KgF) | X > 0.917 KgF | x̅ = 3.05 | PASS |
| | (PpK > 0.91) | Ppk = 1.83 | |
| Extractable | Visual check (no failures) | No Failures | PASS |
| Colour | | | |
| Hematein % | <1.00% (no failures) | No Failures | PASS |

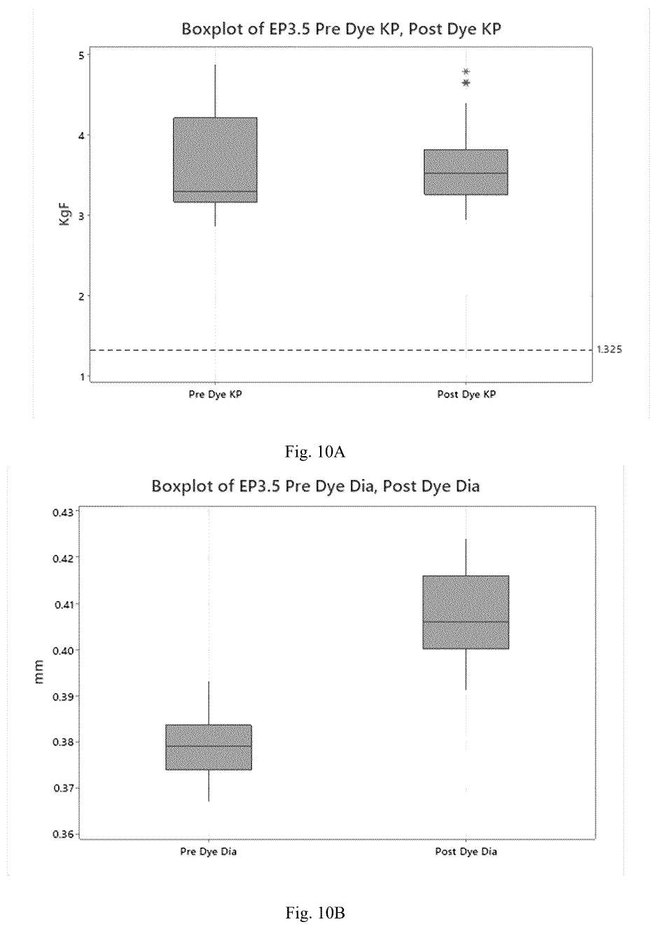

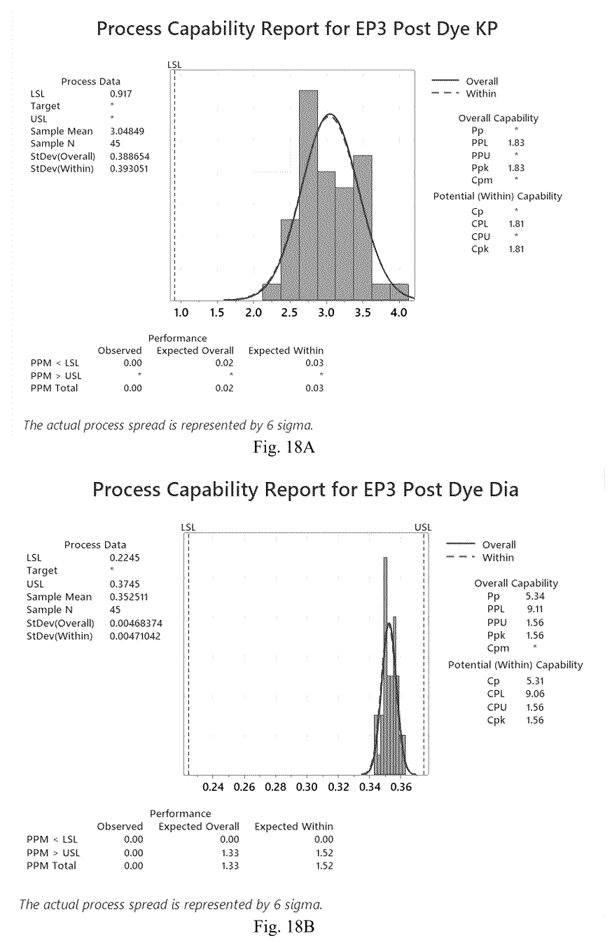

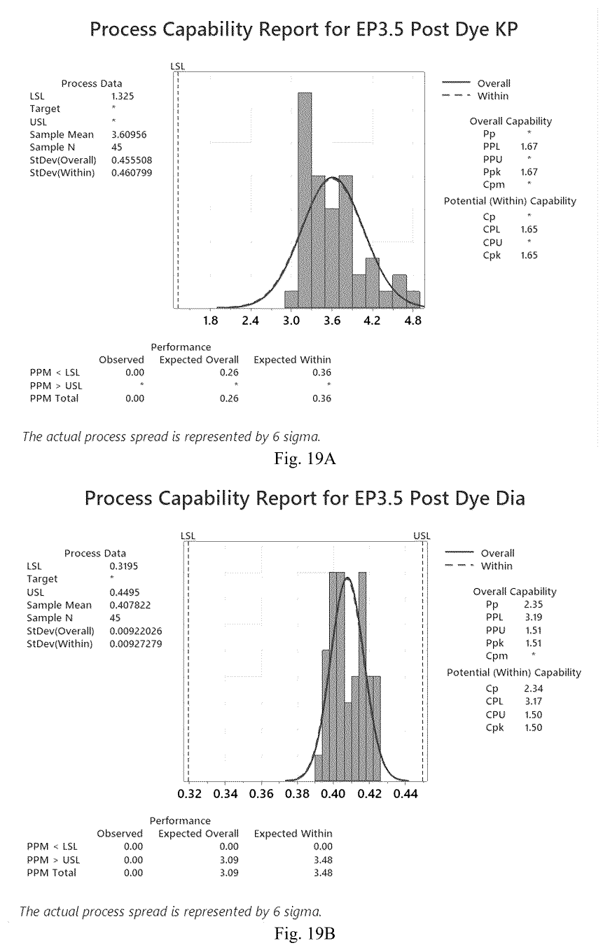

FIGS. 10A and 10B are blox plots of knot pull (KP) strength and diameter (DIA) for EP3.5 (USP 0) nylon suture before and after dyeing. FIGS. 19A and 19B are process capability reports of knot pull strength and diameter for EP3.5 nylon suture after dyeing.

TABLE 7
Part Number: 70A351000 USP 0 (EP 3.5)

| Product | | | |
|---|---|---|---|
| Characteristic | Acceptance Criteria | Results | Pass/Fail |
| Diameter (USP) | 0.3195 ≤ X ≤ 0.4495 | x̅ = 0.4078 | PASS* |
| | (PpK > 0.92) | Ppk = 1.51 | |
| Knot Pull (KgF) | X > 1.325 KgF | x̅ = 3.61 | PASS |
| | (PpK > 0.91) | Ppk = 1.67 | |
| Extractable | Visual check (no failures) | No Failures | PASS |
| Colour | | | |
| Hematein % | <1.00% (no failures) | No Failures | PASS |

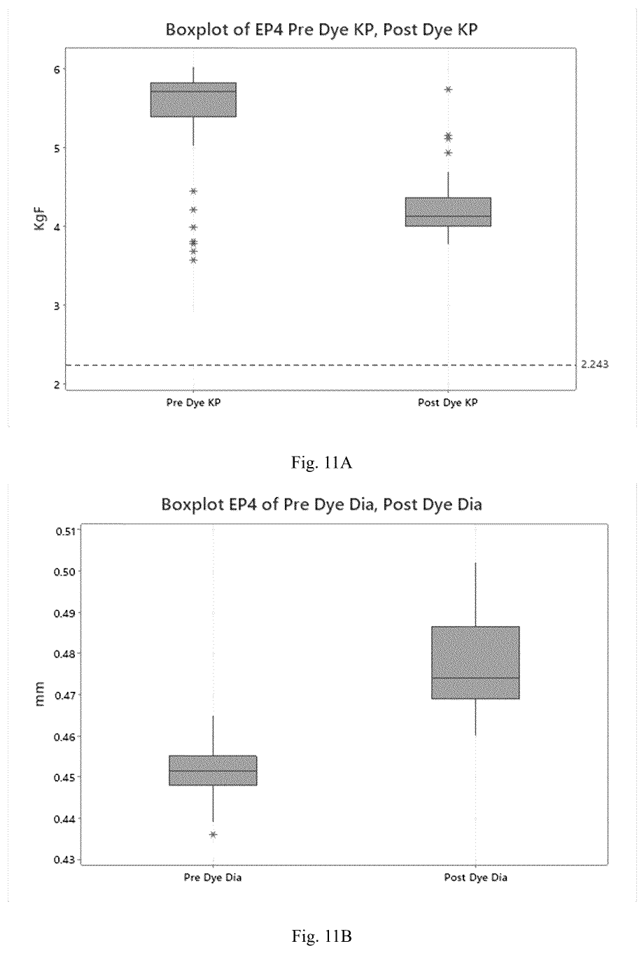

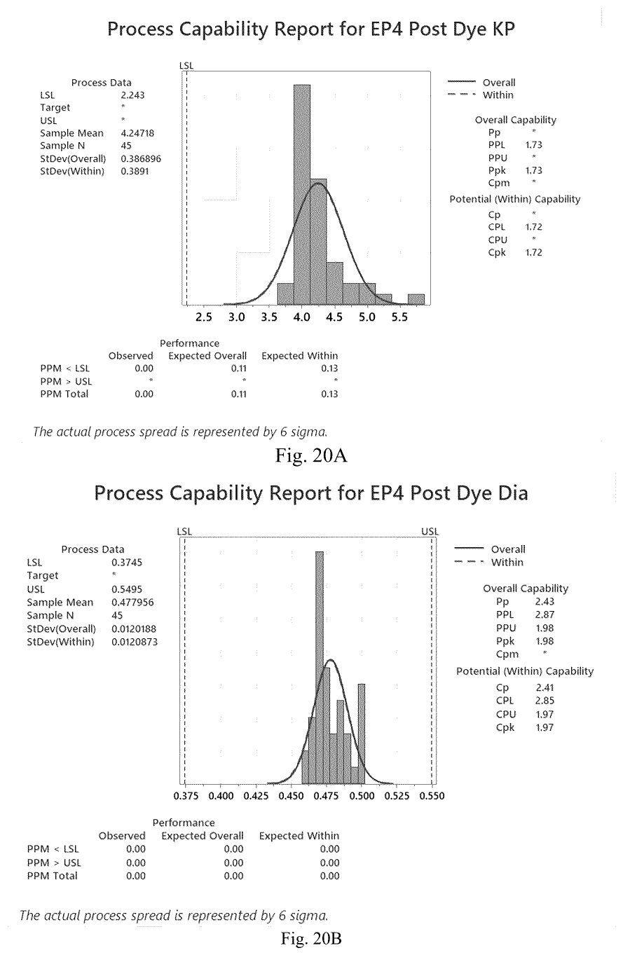

FIGS. 11A and 11B are blox plots of knot pull (KP) strength and diameter (DIA) for EP4 (USP 1) nylon suture before and after dyeing. FIGS. 20A and 20B are process capability reports of knot pull strength and diameter for EP4 nylon suture after dyeing.

TABLE 8
Part Number: 70A401000 USP 1 (EP 4)

| Product | | | |
|---|---|---|---|
| Characteristic | Acceptance Criteria | Results | Pass/Fail |
| Diameter (USP) | 0.3745 ≤ X ≤ 0.5495 | x̅ = 0.478 | PASS |
| | (PpK > 0.92) | Ppk = 1.98 | |
| Knot Pull (KgF) | X > 2.243 KgF | x̅ = 4.25 | PASS |
| | (PpK > 0.91) | Ppk = 1.73 | |
| Extractable | Visual check (no failures) | No Failures | PASS |
| Colour | | | |
| Hematein % | <1.00% (no failures) | No Failures | PASS |

It is noted that the diameter for USP 2-0 and 0 is within the absolute minimum and maximum. However, the mean is higher than the average maximum specification. For USP 2-0, the average min/max is 0.3 to 0.349, however the mean value of the post dye diameter is 0.3525 mm. For USP 0, the average min/max is 0.35 to 0.399, however the mean value of the post dye diameter is 0.4078 mm. These readings have a good capability and results from the pre dye indicate that to post dye the material increased by a consistent 10%. Due to the results observed with the consistent diameter increase between the sizes tested, a further engineering study is conducted to focus on the diameter size and runnage lengths of the material between the pre and post dyeing. For this trial, the extruded material is produced at a lower diameter within specification that should compensate for the diameter increase of the black nylon process keeping the material within specification (e.g., extruded ˜5% lower than the median specification size which should produce product firmly within all specifications). The runnage (metered lengths) of the material pre and post dyeing is measured to observe any deviation in the lengths of each hank size.

The conclusion of this study is that the OQ package for nylon dyed black in skein format (Thies 500-01) is complete and acceptable.

In another study, a performance qualification (PQ) protocol and report (Logwood dyed black nylon (skeins)) verifies that the monofilament Nylon 6.6 dyed black, sizes USP 6-0 to USP 2, using logwood dye on the Thies ECO 500 (THIES 500-01) produces product consistently and effectively when operating at set conditions. The performance qualification protocol includes the process documented in Table 1 herein. The qualification requirements, testing procedures, and acceptance criteria ensure that the monofilament nylon dyed black consistently produces product that meets pre-determined acceptance criteria. The scope of the protocol is limited to monofilament Nylon 6.6 dyed black in skein format using the Thies ECO 500 (THIES 500-01) and its ancillary components and fixtures. The performance qualification determines the method to produce nylon dyed black using hematein dye with Berol at a higher temperature (135° C.).

The performance qualification is run at established nominal conditions discussed herein and also in U.S. Provisional Patent Application No. 63/723,492 Appendix F. Three PQ runs are performed using the full range of materials.

One run is dedicated to USP 2-0 and USP 0 which requires a pre-dye diameter average of a maximum of 0.325 and 0.366 for both sizes respectively to remain within diameter specification post-dye. This is due to the diameter increase discussed above. All other sizes remain within specification post-dye and so do not require a non-standard extrusion size.

Table 9 depicts PQ runs to be completed using 1.25 kg of hematein.

TABLE 9

| | PQ1 | PQ2 | PQ3 |
|---|---|---|---|
| Product Code | (kg) | (kg) | (kg) |
| Nylon Black USP 6-0 / EP 0.7 (70A071000) | 0.5 | 0 | 0 |
| Nylon Black USP 5-0 / EP 1 (70A103000) | 1 | 0 | 0 |
| Nylon Black USP 3-0 / EP 2 (70A203000) | 2.25 | 0 | 0 |
| Nylon Black USP 2-0 / EP 3 (70A302000) | 0 | 1.75 | 1 |
| Nylon Black USP 0 / EP 3.5 (70A351000) | 0 | 2 | 1 |
| Nylon Black USP 2 / EP 5 (70A500500) | 0 | 0 | 1.75 |
| Sum (kg) | 3.5 to 4 | 3.5 to 4 | 3.5 to 4 |

The PQ testing is run to evaluate the following process output/product characteristics at the identified process parameters. These tests and results provide objective evidence that the process parameters used result in products that meet predetermined requirements. Acceptance criteria for this particular testing is shown in Table 10 below including product characteristics for diameter (USP) (AQL 0.078%, sample size 30), knot pull (KgF) (AQL 0.088%, sample size 30), extractable colour (sample size 1), and hematein percentage (sample size 1).

TABLE 10
Acceptance Criteria

| | | Knot Pull | Extractable | |
|---|---|---|---|---|
| Product Code | Diameter (USP) | (KgF) | Colour | Hematein % |
| Nylon Black | 0.0595 ≤ X ≤ 0.1245 | X > 0.061 | Visual check | <1.00% (no |
| USP 6-0/EP0.7 | (PpK > 0.85) | (PpK > 0.84) | (no failures) | failures) |
| (70A071000) | | | | |
| Nylon Black | 0.0845 ≤ X ≤ 0.1745 | X > 0.101 | Visual check | <1.00% (no |
| USP 5-0 / EP 1 | (PpK > 0.85) | (PpK > 0.84) | (no failures) | failures) |
| (70A103000) | | | | |
| Nylon Black | 0.1745 ≤ X ≤ 0.3195 | X > 0.305 | Visual check | <1.00% (no |
| USP 3-0 / EP 2 | (PpK > 0.85) | (PpK > 0.84) | (no failures) | failures) |
| (70A203000) | | | | |
| Nylon Black | 0.2245 ≤ X ≤ 0.3745 | X > 0.509 | Visual check | <1.00% (no |
| USP 2-0 / EP 3 | (PpK > 0.85) | (PpK > 0.84) | (no failures) | failures) |
| (70A302000) | | | | |
| Nylon Black | 0.3195 ≤ X ≤ 0.4495 | X > 1.325 | Visual check | <1.00% (no |
| USP 0 / EP 3.5 | (PpK > 0.85) | (PpK > 0.84) | (no failures) | failures) |
| (70A351000) | | | | |
| Nylon Black | 0.4495 ≤ X ≤ 0.6495 | X > 2.243 | Visual check | <1.00% (no |
| USP 2 / EP 5 | (PpK > 0.85) | (PpK > 0.84) | (no failures) | failures) |
| (70A500500) | | | | |

Diameter and knot pull requirements are determined from United States Pharmacopeia limits. Bath average and absolute requirements are criteria for the purposes of the performance qualification. The post-dye skeins will be fitted onto the swifts to be wound onto a bobbin, before being tested.

Requalification may be pursued if: changes to process result in a new process window outside of the proven window of operation; a new process results through changed environmental conditions; equipment is added or removed from the process flow; the function and intended use of the equipment is changed; a change in raw materials that could affect the performance, quality, safety and effectiveness of the product; equipment is moved or services to the equipment have changed; incoming product is not substantially equivalent to already existing product; and/or the process is determined to be out of control.

The performance qualification report verifies that the monofilament Nylon 6.6 dyed black, sizes USP 6-0 to USP 2, using logwood dye on the Thies ECO 500 (THIES 500-01) produces product consistently and effectively when operating at set conditions. The equipment associated with the performance qualification report is limited to the Logwood Dyed Black Nylon (Thies 500-01) and its ancillary components and fixtures. The products associated with this report are Logwood Dyed Black Nylon (Sizes EP 0.7 to EP 5, USP 6/0 to USP 2).

Results are shown in Table 11. No deviations are observed during execution of the protocol.

TABLE 11

| | | | Extract- | |
|---|---|---|---|---|
| | Diameter | Knot Pull | able | Hema- |
| Product Code | (USP) | (KgF) | Colour | tein % |
| Nylon Black USP 6-0 / | PASS | PASS | PASS | PASS |
| EP 0.7 | x̅ = 0.08865 | x̅ = 0.322 | no failures | no |
| (70A071000) | Ppk = 1.54 | Ppk = 4.09 | | failures |
| Nylon Black USP 5-0 / | PASS | PASS | PASS | PASS |
| EP 1 (70A103000) | x̅ = 0.1362 | x̅ = 0.5606 | no failures | no |
| | Ppk = 2.46 | Ppk = 4.5 | | failures |
| Nylon Black USP 3-0 / | PASS | PASS | PASS | PASS |
| EP 2 (70A203000) | x̅ = 0.246 | x̅ = 1.79 | no failures | no |
| | Ppk = 12.45 | Ppk = 2.67 | | failures |
| Nylon Black USP 2-0 / | PASS | PASS | PASS | PASS |
| EP 3 (70A302000) | x̅ = 0.34 | x̅ = 2.87 | no failures | no |
| | Ppk = 1.95 | Ppk = 3.15 | | failures |
| Nylon Black USP 0 / | PASS | PASS | PASS | PASS |
| EP 3.5 (70A351000) | x̅ = 0.385 | x̅ = 3.454 | no failures | no |
| | Ppk = 5.62 | Ppk = 2.07 | | failures |
| Nylon Black USP 2 / | PASS | PASS | PASS | PASS |
| EP 5 (70A500500) | x̅ = 0.577 | x̅ = 5.59 | no failures | no |
| | Ppk = 3.21 | Ppk = 5.68 | | failures |

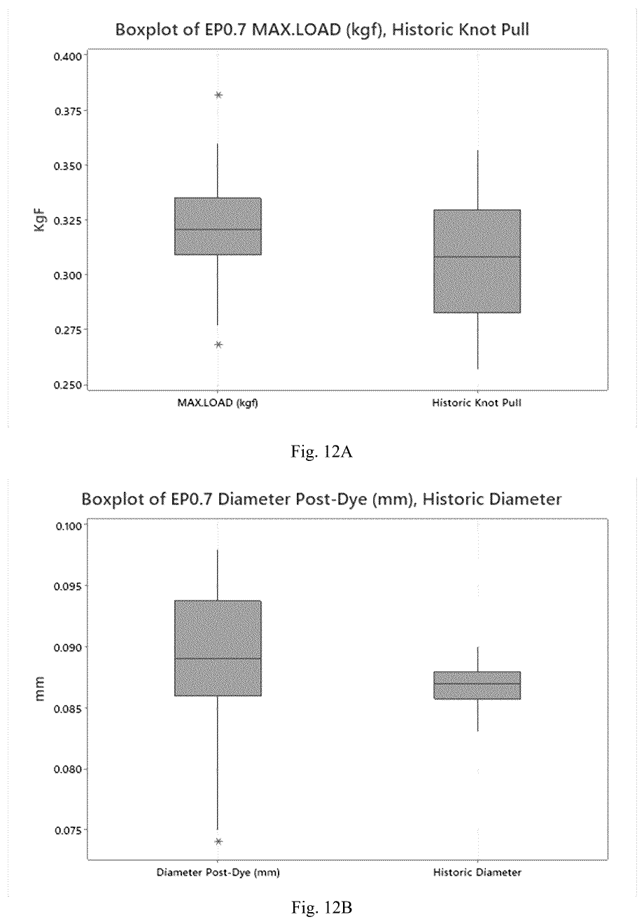

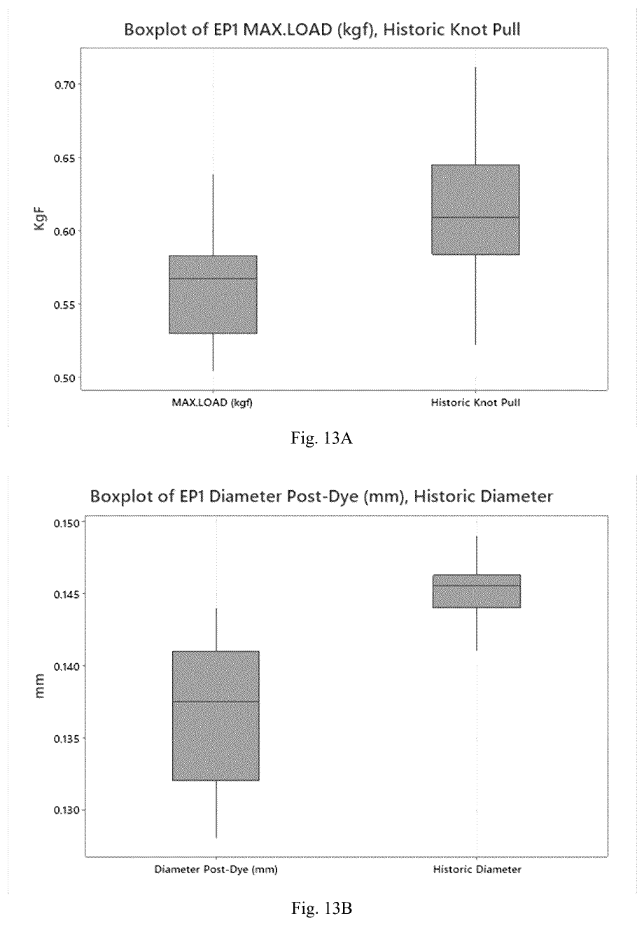

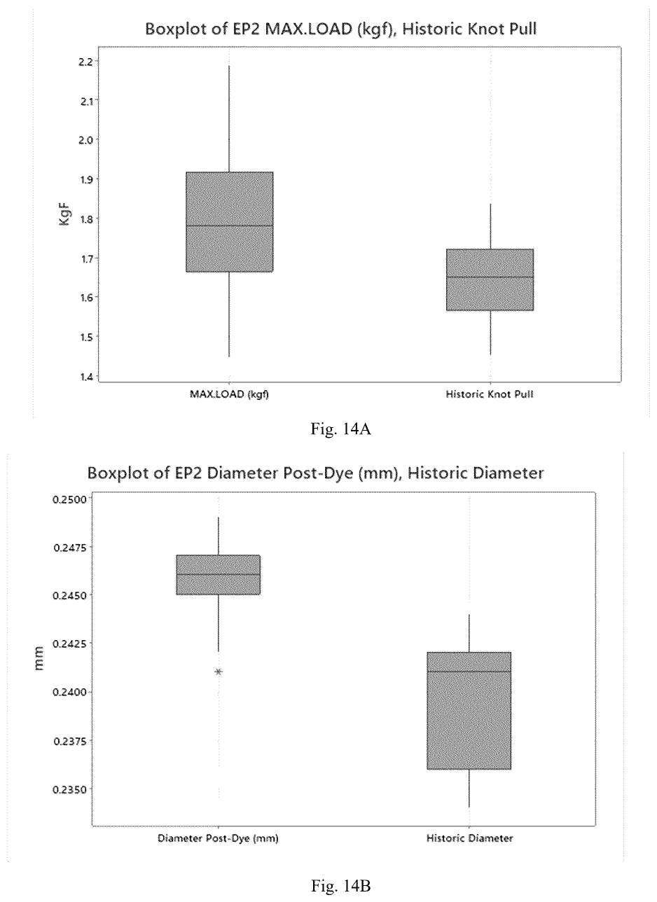

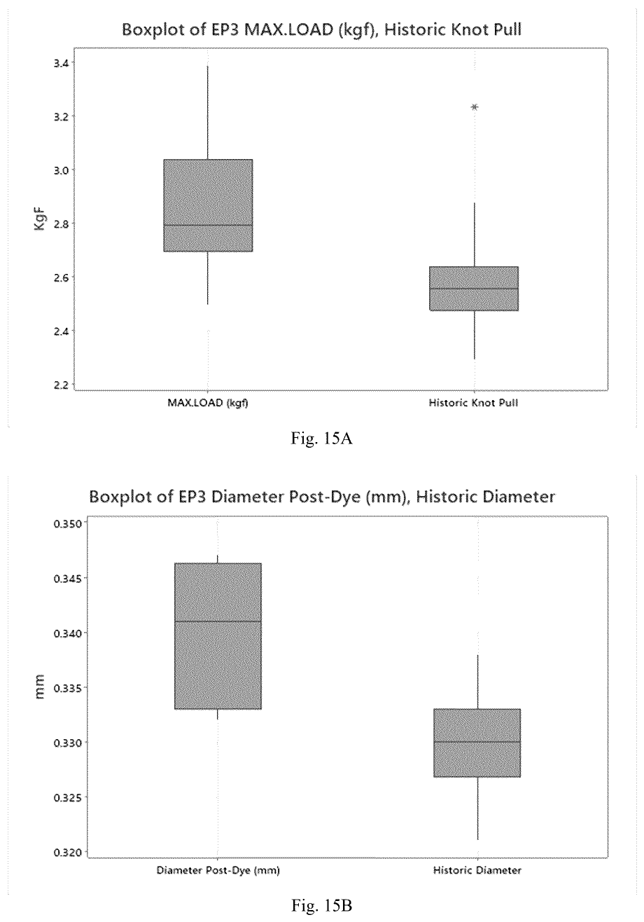

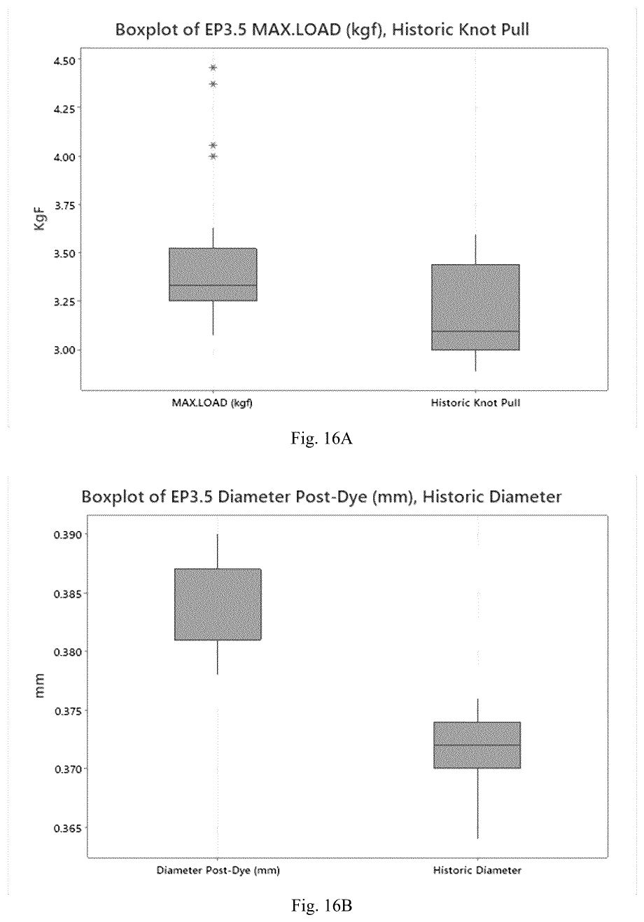

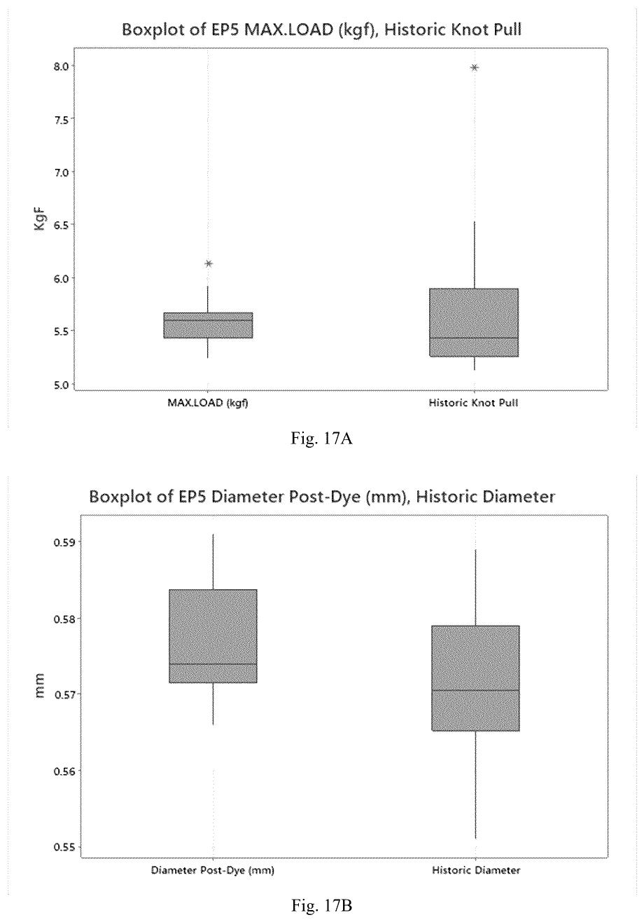

FIGS. 12A and 12B are box plots of knot pull strength (MAX.LOAD kgf) and diameter in mm for EP 0.7 nylon suture after dyeing shown with historic knot pull strength and historic diameter. FIGS. 13A and 13B are box plots of knot pull strength (in kgf) and diameter in mm for EP 1 nylon suture after dyeing shown with historic knot pull strength and historic diameter. FIGS. 14A and 14B are box plots of knot pull strength (in kgf) and diameter in mm for EP 2 nylon suture after dyeing shown with historic knot pull strength and historic diameter. FIGS. 15A and 15B are box plots of knot pull strength (in kgf) and diameter in mm for EP 3 nylon suture after dyeing shown with historic knot pull strength and historic diameter. FIGS. 16A and 16B are box plots of knot pull strength (in kgf) and diameter in mm for EP 3.5 nylon suture after dyeing shown with historic knot pull strength and historic diameter. FIGS. 17A and 17B are box plots of knot pull strength (in kgf) and diameter in mm for EP 5 nylon suture after dyeing shown with historic knot pull strength and historic diameter.

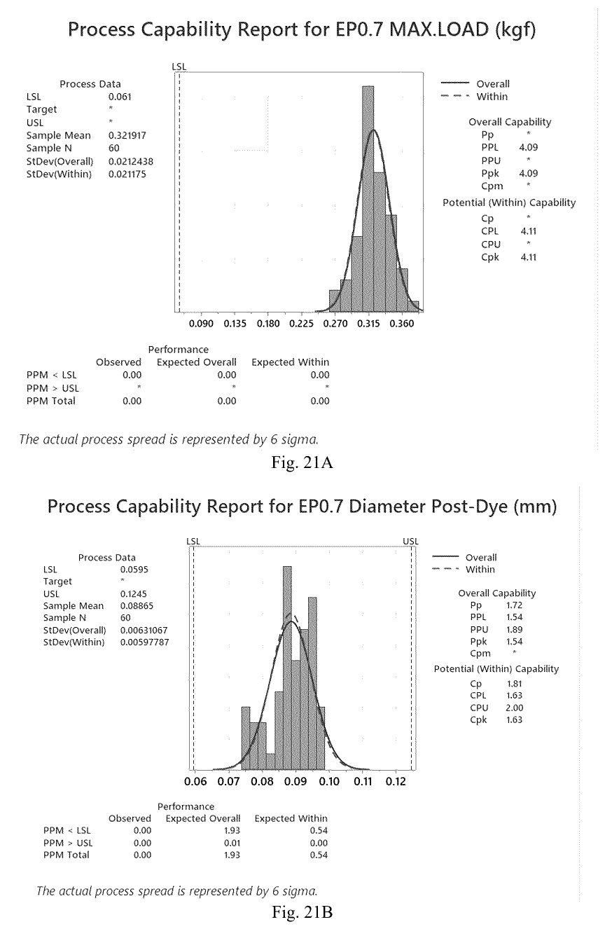

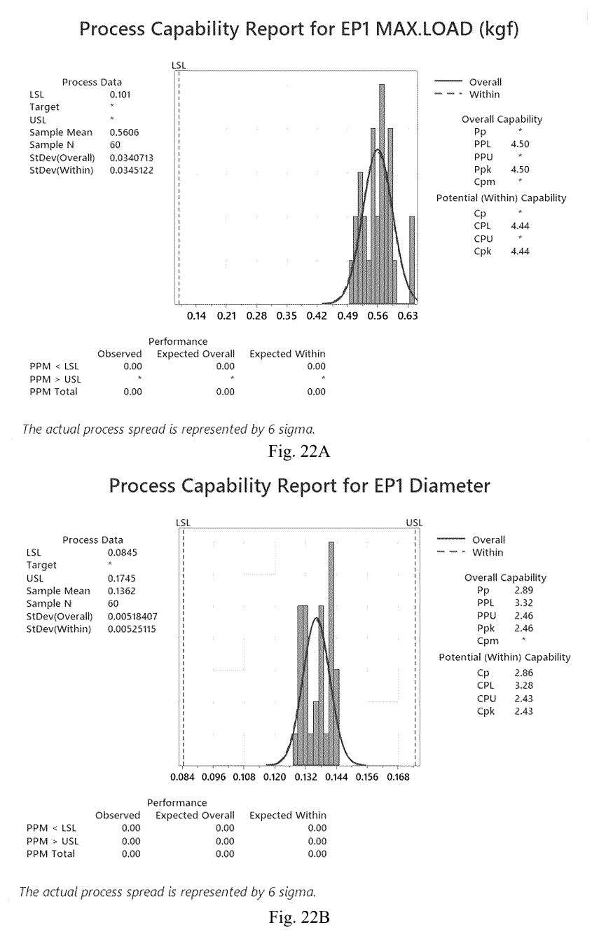

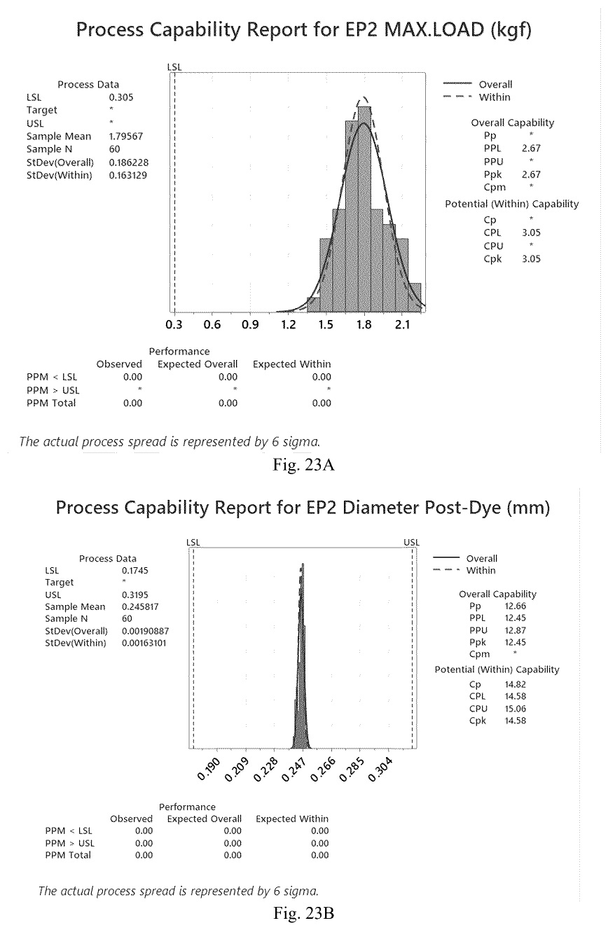

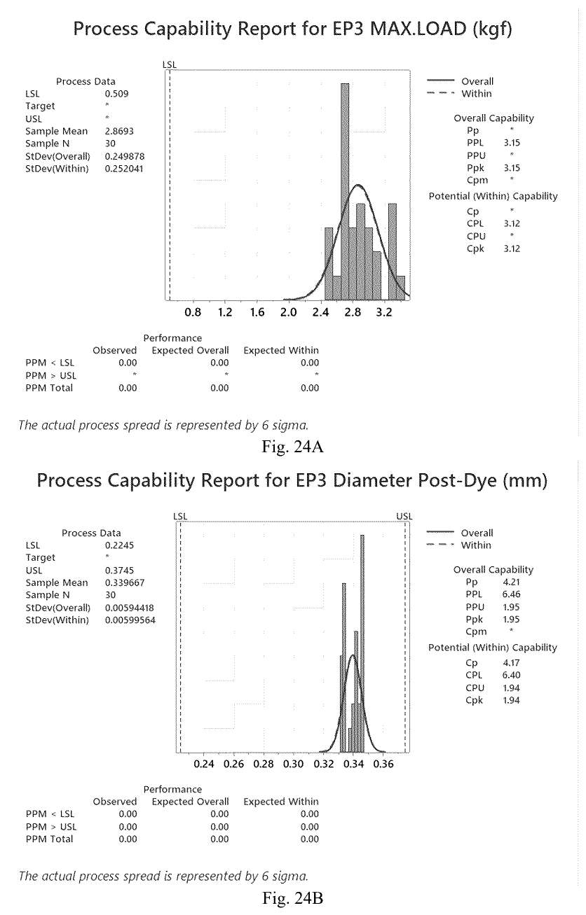

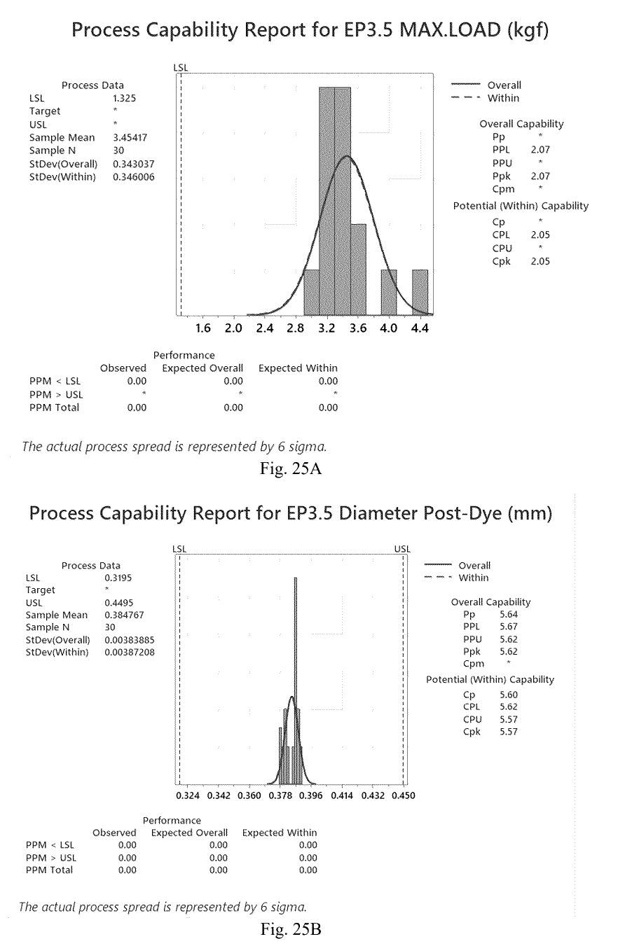

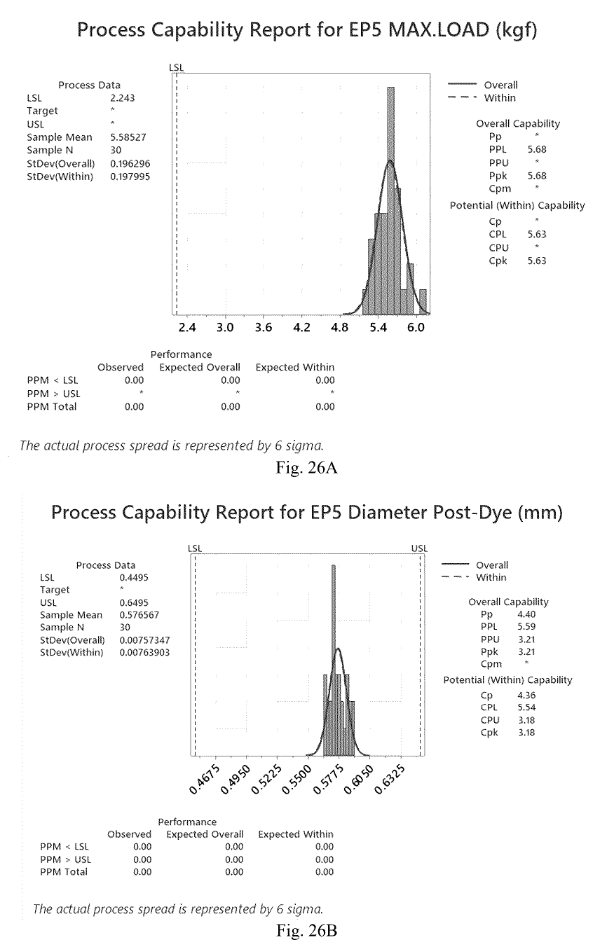

FIGS. 21A and 21B are process capability reports of knot pull strength and diameter for EP 0.7 nylon suture after dyeing. FIGS. 22A and 22B are process capability reports of knot pull strength and diameter for EP 1 nylon suture after dyeing. FIGS. 23A and 23B are process capability reports of knot pull strength and diameter for EP2 nylon suture after dyeing. FIGS. 24A and 24B are process capability reports of knot pull strength and diameter for EP 3 nylon suture after dyeing. FIGS. 25A and 25B are process capability reports of knot pull strength and diameter for EP 3.5 nylon suture after dyeing. FIGS. 26A and 26B are process capability reports of knot pull strength and diameter for EP 5 nylon suture after dyeing.

As discussed above, diameter and knot Pull requirements determined from United States Pharmacopeia limits. Both average and absolute requirements were criteria for all sizes for the purposes of the performance qualification. All results pass the average and absolute requirements for each respective size. It should be noted that sizes EP 3 & EP 3.5 (70A302000 & 70A351000) are extruded at a lower mean value to account for the expected expansion for these sizes.

As discussed above, the post-dye skeins are fitted onto the swifts to be wound onto a bobbin before being tested. It is noted that all sizes are wound without issue except EP0.7, where excessive work was required and heavy waste was observed on unwound material. While the discrete data results for EP0.7 were successful and within specification, this rework required would result in a non-ideal production scenario. However, production improvements are known to those skilled in the art.

It is concluded that the performance qualification package for Logwood Dyed Black Nylon (Thies 500-01) is complete and acceptable and the qualification was performed according to protocol.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for dyeing suture material, the method comprising the steps of:
a) mixing hematein and a nonionic surfactant to obtain a dye solution;
b) placing a suture material in H₂O in a vessel;
c) heating the H₂O in the vessel from 20° C. to 40° C.;
d) adding the dye solution to the heated H₂O in the vessel to form a staining bath;
e) incubating the suture material in the staining bath at 40° C. to 135° C. for 120 min to 150 min, thereby obtaining dyed suture material,
wherein the method is carried out in the absence of chromium salts.

2. The method of claim 1, further comprising the steps of:
f) removing the staining bath from the vessel;
g) filling the vessel with H₂O at a temperature of 20° C. to 60° C.;
h) adding NaOH to the H₂O in the vessel;
i) maintaining the temperature in the vessel at 40° C. to 60° C. for a period of 30 min to 45 min;
j) draining the vessel;
k) washing the dyed suture material with H₂O; and
l) drying the dyed suture material.

3. A dyed suture material produced by the method of claim 1.

4. A surgical suturing kit comprising the dyed suture material of claim 3 and one or more suture needles or other medical devices in a sterilizable package having a plurality of discrete compartments.

5. The surgical suturing kit of claim 4, wherein the dyed suture is nylon having a size of USP 6/0 to USP 2.

6. A method for dyeing suture material with a pressure dyer, the method comprising:
incubating the suture material in a dye solution comprising hematein in a vessel at a high temperature for at least 45 minutes
wherein the method is carried out in the absence of chromium salts.

7. The method of claim 6, wherein the dye solution further comprises a nonionic surfactant.

8. The method of claim 6, wherein the high temperature is at least 110° C.

9. The method of claim 6, wherein the high temperature is between about 110° C. and 140° C.

10. The method of claim 6, wherein the suture material is silk, nylon, or polyester.

11. The method of claim 10, wherein the suture material is monofilament nylon or multifilament nylon.

12. The method of claim 6, wherein the suture material is in the form of a skein or a roll.

13. The method of claim 6, wherein 0.1 kg to 4 kg of the suture material is placed in the vessel.

14. The method of claim 7, wherein the nonionic surfactant is selected from the group consisting of an ethoxylated castor oil, a polysorbate, a sorbitan ester, a polyoxyethylene alkyl ether, an octylphenol ethoxylate, a block copolymer of ethylene oxide and propylene oxide, an ethoxylated fatty alcohol, and a nonylphenol ethoxylate.

15. The method of claim 7, wherein the nonionic surfactant is PEG-36 castor oil.

16. The method of claim 7, wherein in a weight to volume ratio between the hematein and the nonionic surfactant is 2.5 kg/L to 12.5 kg/L.

17. The method of claim 6, wherein a weight ratio between the hematein and the suture material is 1:1 to 1:6.

18. The method of claim 7, wherein the dye solution further comprises at least 4 to 15 volumes of H₂O.

19. The method of claim 6, wherein the incubating is carried out by heating the staining bath from 40° C. to 135° C. over a 30 min period and holding the temperature at 135° C. for 60 min to 120 min.

20. The method of claim 6 further comprising washing the suture material in water at a first temperature less than the incubation temperature and further subjecting the suture material to at least one of the following:
a solution comprising sodium hydroxide and/or
a first water rinse at a second temperature greater than the first temperature and a second water rinse at a temperature less than the second temperature.

21. A dyed suture material produced by the method of claim 6.

22. A dyed suture material of claim 21 wherein the dyed suture material contains less than 1% hematein, achieves sufficient suture strength, and/or achieves sufficient colour fastness.

23. A surgical suturing kit comprising the dyed suture material of claim 21.

24. The surgical suturing kit of claim 23, wherein the dyed suture is nylon having a size of USP 6/0 to USP 2.

25. A method for dyeing suture material comprising:
loading the suture material in a vessel in a pressure dyer;
adding a staining bath comprising hematein and a nonionic surfactant;
increasing the temperature within the vessel to a high temperature;
incubating the suture material within the staining bath for at least 45 minutes; and
draining the staining bath
wherein the method is carried out in the absence of chromium salts.

26. The method of claim 25, wherein the high temperature is at least 110° C.

27. The method of claim 25, wherein 0.1 kg to 4 kg of the suture material is placed in the vessel.

28. The method of claim 25, wherein the nonionic surfactant is selected from the group consisting of an ethoxylated castor oil, a polysorbate, a sorbitan ester, a polyoxyethylene alkyl ether, an octylphenol ethoxylate, a block copolymer of ethylene oxide and propylene oxide, an ethoxylated fatty alcohol, a nonylphenol ethoxylate, and PEG-36 castor oil.

29. The method of claim 25, wherein in a weight to volume ratio between the hematein and the nonionic surfactant is 2.5 kg/L to 12.5 kg/L.

30. The method of claim 25 further comprising at least one of the following steps: washing the suture material in water at a temperature that is lower than the incubation temperature or incubating the suture material in a scouring solution.

31. A dyed suture material produced by the method of claim 25.

32. A dyed suture material of claim 31 wherein the dyed suture material contains less than 1% hematein, achieves sufficient suture strength, and/or achieves sufficient colour fastness.

33. A surgical suturing kit comprising the dyed suture material of claim 31.

34. The surgical suturing kit of claim 33, wherein the dyed suture is nylon having a size of USP 6/0 to USP 2.
